SEQUENCE LISTING (21 sequences)

SEQ ID NO: 1 - DNA, 270 bp, Homo sapiens
tggtaccatccggacaaagcctgcgcgcgccccgccccgccattggccgtaccgccccgcgccgccgccccatcccgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcg

SEQ ID NO: 2 - DNA, 239 bp, Homo sapiens
cgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgc

SEQ ID NO: 3 - DNA, 245 bp, Homo sapiens
ccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 4 - DNA, 2751 bp, Human adenovirus
catcatcaataaatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtgaccggtggtaccatccggacaaagcctgcgcgcgccccgccccgccattggccgtaccgccccgcgccgccgccccatcccgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcgaccggtgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggaggcgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaatttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacgtcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatggtcgaccggtaccgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcaccggtcgacgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcgg

SEQ ID NO: 5 - DNA, 38 bp, Artificial Sequence
  Description of Artificial Sequence Synthetic primer
catctgcagcatgaagcgcgcaagaccgtctgaagata

SEQ ID NO: 6 - DNA, 58 bp, Artificial Sequence
  Description of Artificial Sequence Synthetic primer
  modified_base (23): Inosine
cgttgaaacataacacaaacgantctttattcatcttctctaatataggaaaaggtaa

SEQ ID NO: 7 - DNA, 58 bp, Artificial Sequence
  Description of Artificial Sequence Synthetic primer
ttaccttttcctatattagagaagatgaataaagaatcgtttgtgttatgtttcaacg

SEQ ID NO: 8 - DNA, 30 bp, Artificial Sequence
  Description of Artificial Sequence Synthetic primer
agacaagcttgcatgcctgcaggacggagc

SEQ ID NO: 9 - protein, 4 aa, Human adenovirus type 5
KKTK

SEQ ID NO: 10 - protein, 9 aa, Human adenovirus type 5
KLGTGLSFD

SEQ ID NO: 11 - protein, 19 aa, Human adenovirus type 5
GNLTSQNVTTVSPPLKKTK

SEQ ID NO: 12 - protein, 17 aa, Human adenovirus type 35
GTLQENIRATAPITKNN

SEQ ID NO: 13 - DNA, 972 bp, Human adenovirus type 35
atgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacgaataa

SEQ ID NO: 14 - protein, 323 aa, Human adenovirus type 35
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDE

SEQ ID NO: 15 - DNA, 1746 bp, Human adenovirus type 5
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 16 - protein, 581 aa, Human adenovirus type 5
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 17 - DNA, 1773 bp, Human adenovirus
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacgaataa

SEQ ID NO: 18 - protein, 590 aa, Human adenovirus
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDE

SEQ ID NO: 19 - DNA, 1330 bp, Human adenovirus type 3
ggccttcgagacctcctacccatgaactaatcattgcccctaccttacccaatcaaaatattaataaagacacttacttgaaatcagcaatacagtctttgtcaaaactttctaccagcagcacctcaccctcttcccaactctggtactctaaacgtcggagggtggcatactttctccacactttgaaagggatgtcaaattttatttcctcttctttgcccacaatcttcatttctttatccccagatggccaagcgagctcggctaagcacttccttcaacccggtgtacccttatgaagatgaaagcagctcacaacacccatttataaatcctggtttcatttcccctgacgggttcacacaaagtccaaacggggttttaagtcttaaatgtgttaatccacttaccactgcaagcggctccctccaacttaaagtgggaagtggtcttacagtagacactactgatggatccttagaagaaaacatcaaagttaacacccccctaacaaagtcaaaccattctataaatttaccaataggaaacggtttgcaaatagaacaaaacaaactttgcagtaaactcggaaatggtcttacatttgactcttccaattctattgcactgaaaaataacactttatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactgacaacaaaaataaagttcaacattttttattgaaattccttttacagtattcgagtagttattttgcctcccccttcccatttaacagaatacaccaatctctccccacgcacagctttaaa

SEQ ID NO: 20 - protein, 319 aa, Human adenovirus type 3
MAKRARLSTSFNPVYPYEDESSSQHPFINPGFISPDGFTQSPNGVLSLKCVNPLTTASGSLQLKVGSGLTVDTTDGSLEENIKVNTPLTKSNHSINLPIGNGLQIEQNKLCSKLGNGLTFDSSNSIALKNNTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 21 - protein, 323 aa, Human adenovirus type 35
MTKRVRLSDSFNPVYPYEDESTSQHPFYNPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

(19) United States
(12) Patent Application Publication
(10) Pub. No.: US 2007/0010016 A1
(43) Pub. Date: Jan. 11, 2007

(54) Gene transfer with adenoviruses having modified fiber proteins

(76) Inventors: Alan McCelland, Danville, CA (US); Susan Stevenson, Clarksburg, MD (US); Mario Gorziglia, Doleystown, PA (US); Elio Vanin, Memphis, TN (US)

Correspondence Address: DLA Piper Rudnick Gray Cary LLP;Suite 800, 153 Townsend Street, San Francisco, CA, 94107-1957, US

(21) Appl. No.: 11/371,488

(22) Filed: Mar. 9, 2006

Related U.S. Application Data
(60) Provisional application No. 60/660,333, filed on Mar. 11, 2005.

(51) Int. Cl. C12N 15/861; C12N 7/00

(52) U.S. Cl. 435/456; 435/235.1; 977/802

(57) ABSTRACT

Methods and compositions for transducing tumor cells using adenoviral vectors which comprise: a chimeric or modified adenovirus fiber protein and the coding sequence for a therapeutic agent, are provided. The chimeric or modified adenovirus fiber protein has at least a portion of an adenovirus fiber shaft of a first serotype and at least a portion of an adenovirus fiber head of a second serotype wherein the adenovirus comprising such a chimeric or modified adenovirus fiber protein exhibits enhanced transduction of tumor cells.

### CROSS-REFERENCE TO RELATED APPLICATIONS

This application claims priority benefit of U.S. Patent Application No. 60/660,333, filed Mar. 11, 2005, the contents of which is hereby incorporated by reference in it's entirety.

### FIELD OF THE INVENTION

The present invention relates to adenoviral vectors which comprise a modified or chimeric fiber protein and exhibit enhanced transduction of tumor cells.

### BACKGROUND OF THE TECHNOLOGY

Adenovirus genomes are linear, double-stranded DNA molecules about 36 kilobase pairs long. Each extremity of the viral genome has a short sequence known as the inverted terminal repeat (or ITR), which is necessary for viral replication. The well-characterized molecular genetics of adenovirus render it an advantageous vector for gene transfer. The knowledge of the genetic organization of adenoviruses allows substitution of large fragments of viral DNA with foreign sequences. In addition, recombinant adenoviruses are stable structurally, and no rearranged viruses are observed after extensive amplification.

Adenoviruses may be employed as delivery vehicles for introducing desired genes into eukaryotic cells. The adenovirus delivers such genes to eukaryotic cells by binding cellular receptors. The adenovirus fiber protein is responsible for such attachment. (Philipson, et al., J. Virol., Vol. 2, pgs. 1064-1075 (1968)). The fiber protein includes a tail region, a shaft region, and a globular head region which contains the putative receptor binding region. The fiber spike is a homotrimer, and there are 12 spikes per virion.

In susceptible cells, the adenoviral cellular entry pathway is an efficient process which involves two separate cell surface events (Wickham et al., Cell, Vol. 73, pgs, 309-319 (1993)). First, a high affinity interaction between the adenoviral capsid fiber protein and a cell surface receptor (e.g. CAR or CD46) mediates the attachment of the adenoviral particle to the cell surface. A subsequent association of the penton with the cell surface integrins, αvβ3 and αvβ5 which act as co-receptors, potentiate virus internalization (Wickham, 1993). Competition binding experiments using intact adenoviral particles and expressed fiber proteins have provided evidence for the existence of at least two distinct adenoviral fiber receptors which interact with the subgenus B (Adenovirus 3) and subgenus C (Adenovirus 5) adenoviruses (Defer, et al., J. Virol., Vol. 64, 3661-3673 (1990); Mathias, et al., J. Virol., Vol. 68, pgs. 6811-6814 (1994); Stevenson, et al., J. Virol., Vol. 69, pgs. 2650-2857 (1995)). Although Adenovirus 5 and Adenovirus 3 utilize different fiber binding receptors, αv integrins enhance entry of both serotypes into cells (Mathias, 1994). This suggests that the binding and entry steps are unlinked events and that fiber attachment to various cell surface molecules may permit productive entry. It is likely that additional receptors exist for other adenoviral serotypes although this remains to be demonstrated. Adenoviral vectors derived from the human Subgenus C, Adenovirus 5 serotype are efficient gene delivery vehicles which readily transduce many nondividing cells. Adenoviruses infect a broad range of cells and tissues including lung, liver, endothelium, and muscle (Trapnell, et al. Curr. Opinion Biotech., Vol. 5, pgs. 617-625 (1994). High titer stocks of purified adenoviral vectors can be prepared which makes the vector suitable for in vivo administration. Various routes of in vivo administration have been investigated including intravenous delivery for liver transduction and intratracheal instillation for gene transfer to the lung. As the adenoviral vector system is more widely applied, it is becoming apparent that some cell types may be refractory to recombinant adenoviral infection. Both the fiber binding receptor and αvβ3 and αvβ5 integrins are important for high efficiency infection of target cells. Efficient transduction requires fiber mediated attachment as demonstrated by the effectiveness of recombinant soluble fiber in blocking gene transfer (Goldman, et al., J. Virol., Vol. 69, pgs. 5951-5958 (1995)). Transduction of cells which lack fiber receptors occurs with much lower efficiency and requires high multiplicities of input vector (Freimuth, et al., J. Virol., Vol. 70, pgs. 4081-4085 (1996); Haung, et al., J. Virol., Vol. 70, pgs. 4502-4508 (1996)). Fiber independent transduction likely occurs through direct binding of the penton base arginine-glycine-aspartic acid, or RGD, sequences to cell surface integrins. Blockade of the RGD:integrin pathway reduces gene transfer efficiencies by several fold (Freimuth, 1996; Haung, 1996), but the effect is less complete than blockade of the fiber receptor interaction, suggesting that the latter is more critical.

Low level gene transfer may result from a deficiency in one of the components of the entry process in the target cell. For example, inefficient gene transfer to human pulmonary epithelia has been attributed to a deficiency in αvβ5 integrins (Goldman, 1995). Other cell types such as vascular endothelial and smooth muscle cells have been identified as being deficient in fiber dependent transduction due to a low level of the Adenovirus 5 receptor (Wickham, et al., J. Virol., Vol. 70, pgs. 6831-6838 (1996)). Several approaches have been undertaken to target adenoviral vectors to improve or enable efficient transduction of target cells. These strategies include alteration of the penton base to target selectively specific cell surface integrins (Wickham, et al., Gene Ther., Vol. 2, pgs. 750-756 (1995); Wickham, et al., J. Virol., Vol. 70, pgs. 6831-6838 (1996)) and modification of the fiber protein with an appropriate ligand to redirect binding (Michael, et al., Gene Ther., Vol. 2, pgs. 660-668 (1995); Stevenson, 1995).

### SUMMARY OF THE INVENTION

The present invention relates to improved adenoviral vectors comprising modified fiber proteins such that prior to modification of the adenovirus is of a first serotype, and the adenovirus is modified such that at least a portion, preferably the head region, of the fiber of the adenovirus of the first serotype is removed and replaced with at least a portion, preferably the head region, of the fiber of an adenovirus of a second serotype.

This invention also relates to gene delivery or gene transfer vehicles other than adenoviruses, which have been modified to include at least a portion, preferably the head region, of the fiber of an adenovirus of a desired serotype, whereby the gene delivery or gene transfer vehicle will bind to a receptor for the region of the fiber, preferably the head region, of the adenovirus of the desired serotype. Such gene delivery or gene transfer vehicles may be viruses, such as, for example, retroviruses, adeno-associated virus, and Herpes viruses, which have a viral surface protein which has been modified to include at least a portion of the fiber, preferably the head region, of the fiber of an adenovirus of a desired serotype. Alternatively, the gene delivery or gene transfer vehicle may be a non-viral gene delivery or gene transfer vehicle, such as a plasmid, to which is bound at least a portion, preferably the head region, of the fiber of an adenovirus of a desired serotype. In another example, the gene delivery or gene transfer vehicle may be a proteoliposome which encapsulates an expression vehicle, wherein the proteoliposome includes a portion, preferably the head region, of the fiber of an adenovirus of a desired serotype.

This invention further relates to adenoviruses of the Adenovirus 35 serotype which include at least one heterologous DNA sequence, and to the transfer of polynucleotides into cells which include a receptor which binds to the head region of the fiber of Adenovirus 35, by contacting such cells with a gene transfer vehicle which includes the head region of the fiber of Adenovirus 35.

The present invention is directed to the transduction of cells with adenoviruses wherein at least a portion of the fiber of the adenovirus, and in particular the head region, is removed and replaced with a fiber portion, and in particular, a head region of the fiber, having novel receptor specificities. Binding of recombinant Adenovirus 5 and Adenovirus 35 fiber proteins to cellular receptors has been examined previously, and it was demonstrated that the receptor specificity of the fiber protein can be altered by exchanging the head domains between these two fiber proteins (Stevenson, 1995). Thus, the present invention is directed to the transduction of cells with a modified adenovirus having a chimeric fiber, wherein the adenovirus, prior to modification, is of a first serotype, and the adenovirus is modified such that at least a portion of the fiber, and in particular the head region, of the adenovirus is removed and replaced with at least a portion of the fiber of an adenovirus of the second serotype. Applicants have found that such adenoviruses bind to cells having a receptor for the adenovirus of the second serotype. Applicants also have found that such adenoviruses may bind to cells which are refractory to adenoviruses of the first serotype, yet are bound by the modified adenoviruses through the binding of the head region of the fiber of the modified adenovirus to a receptor for the adenovirus of the second serotype.

The present invention also is directed to gene delivery or gene transfer vehicles, other than adenoviruses, which include at least a portion, preferably the head region, of the fiber of an adenovirus of a desired serotype. Such gene transfer vehicles are useful for delivering polynucleotides to cells which have a receptor that binds to the fiber of the adenovirus of a desired serotype. The gene transfer vehicles which may be employed include, but are not limited to, retroviruses, adeno-associated virus, Herpes viruses, plasmids which are linked chemically to the at least a portion of the fiber of the adenovirus of a desired serotype, and proteoliposomes encapsulating the polynucleotide which is to be transferred into cells.

In yet another embodiment, the present invention is directed to an adenovirus of the Adenovirus 35 serotype which includes at least one heterologous DNA sequence, preferably encoding a cytokine.

In a further embodiment, the present invention also is directed to the transfer of polynucleotides into cells which include a receptor for Adenovirus 35 by contacting such cells with a gene transfer vehicle including at least a portion, and preferably the head region, of the fiber of Adenovirus 35.

### BRIEF DESCRIPTION OF THE FIGURES

FIG. 1A shows the results of genomic analysis of the wild type fiber, Av1LacZ4 and chimeric fiber, Av9LacZ4 adenoviral vectors. FIG. 1A shows ScaI (S), DraI (D), EcoRI (E) and BamHI (B) restriction endonuclease sites on a schematic diagram for each vector. The predicted DraI and ScaI restriction fragments and the expected sizes for Av1LacZ4 and Av9LacZ4 are highlighted. DNA was isolated from each vector, digested with the indicated restriction endonucleases, and Southern blot analysis carried out using standard procedures.

FIG. 1B shows digested DNA samples (0.4 ug) that were applied to a 0.8% agarose gel and stained with ethidium bromide to visualize the individual DNA fragments. The combined .lambda.DNA/HindIII and .phi.X174 RF DNA/HaeIII DNA size markers (M) are indicated. The Av1LacZ4 wildtype vector was digested with: lane 1, ScaI; lane 2, DraI; and lane 3, EcoRI and BamHI. The Av9LacZ4 chimeric fiber vector was digested with: lane 4, ScaI; lane 5, DraI and lane 6, EcoRI and BamHI.

FIG. 1C shows digested DNA fragments as shown in FIG. 1B that were transferred to a Zetaprobe membrane and hybridized with the [³²P]-labeled 500 bp Adenovirus 3 fiber head domain probe at approximately 1×10⁶ cpm/ml and exposed to film for 12 hours. The expected fragments derived from Av9LacZ4 which hybridized with the Adenovirus 3 fiber head probe are indicated.

FIGS. 2A and B show the results of Western immunoblot analysis of adenoviral capsid proteins. An equivalent number of adenoviral particles for the Av1LacZ4 (lanes 1 and 4), Av9LacZ4 (lanes 2 and 5) vectors or a control virus containing the full length Adenovirus 3 fiber protein (lanes 3 and 6) were subjected to 4/15% SDS PAGE and Western blot analysis under denaturing conditions. (A) 2×10¹⁰ adenoviral particles were applied per lane and the membrane was developed with the anti-fiber monoclonal antibody, 4D2-5 and an anti-mouse IgG-HRPO conjugated secondary antibody by chemiluminescence. (B) 6×10¹⁰ particles were applied per lane and the membrane was developed using a rabbit anti-Adenovirus 3 fiber specific polyclonal antibody and donkey anti-rabbit IgG-HRPO secondary antibody by chemiluminescence. The positions of molecular weight markers are indicated.

FIGS. 3A and 3B are graphs of the results of competition viral transduction assays. HeLa cell monolayers were incubated with increasing concentrations of purified Adenovirus 5 fiber trimer protein (5F, FIG. 3A) or with an insect cell lysate containing the Adenovirus 3 fiber protein (3F/CL, FIG. 3B) prior to transduction with 100 total particles per cell of either the Av1LacZ4 (open circles) or Av9LacZ4 (closed circles) adenoviral vectors. After 24 hours, the cells were analyzed for β-galactosidase expression as described in Example 1. The percentage of adenoviral transduction at each concentration of competitor is plotted. Each point is the average .+/−. standard deviation of three independent determinations for a representative experiment.

FIGS. 4 A-F show differential adenoviral-mediated transduction properties of human cell lines. HeLa (FIGS. 4A and 4B), MRC-5 (FIGS. 4C and 4D), and FaDu (FIGS. 4E and 4F) cells were transduced with the Av1LacZ4 (FIGS. 4A, 4C, and 4E) or Av9LacZ4 (FIGS. 4B, 4D, and 4F) vectors at 1000 total particles per cell. After 24 hours the cells were analyzed for β-galactosidase expression as described in Example 1. Representative photomicrographs are shown.

FIGS. 5A, 5B, and 5C are graphs showing Adenoviral-mediated transduction properties of HeLa, MRC-5, and FaDu human cell lines. The indicated cells were transduced with 0,10,100, and 1000 total particles per cell of the Av1LacZ4 (open circles) or Av9LacZ4 (closed circles) vectors for one hour at 37C. in a total volume of 0.2 ml of culture medium. After 24 hours, the cells were fixed and stained with X-gal as described in Example 1. The percent transduced cells per high power field was determined for each vector dose. The data represent the average percent transduction .+−. standard deviation for three independent experiments and each vector dose was carried out in triplicate. The percentage transduction of HeLa (FIG. 5A), MRC-5 (FIG. 5B) and FaDu (FIG. 5C) cells at each vector dose is displayed.

FIGS. 6A and 6B are graphs showing differential adenoviral-mediated transduction properties of human cell lines. The percent transduction efficiency for each cell line infected with the Av1LacZ4 (open bars) or Av9LacZ4 (closed bars) vectors is displayed for the vector dose of 100 (FIG. 6A) and 1000 (FIG. 6B) particles per cell. The data represent the mean .+−. standard deviation from three independent experiments. The cell lines are as follows: HeLa: human cervical carcinoma cells; HDF: human diploid fibroblasts; THP-1: human monocytes; MRC-5: human embryonic lung diploid fibroblasts; FaDu: human squamous carcinoma cells; HUVEC: human umbilical vein endothelial cells, and HCAEC: human coronary artery endothelial cells.

FIG. 7 is a graph illustrating the anti-tumor efficacy of OV1991 in the FaDu human head and neck tumor xenograft tumor model.

FIG. 8 is a graph illustrating the anti-tumor efficacy of Ad5/Ad35 and Ad5/Ad3 chimeric fiber vectors in the A375-luc human melanoma xenograft tumor model.

### DETAILED DESCRIPTION OF THE INVENTION

Definitions

Unless otherwise indicated, all terms used herein have the same meaning as they would to one skilled in the art and the practice of the present invention will employ, conventional techniques of microbiology and recombinant DNA technology, which are within the knowledge of those of skill of the art.

The abbreviation “pfu” stands for plaque forming units.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo. For purposes of the present invention, these terms refer to adenoviruses, including recombinant adenoviruses formed when an adenoviral vector of the invention is encapsulated in an adenovirus capsid.

An “adenovirus vector” or “adenoviral vector” (used interchangeably) as referred to herein is a polynucleotide construct which can be packaged into an adenoviral virion. In some embodiments, an adenoviral vector of the invention includes a therapeutic gene sequence or transgene, such as a cytokine gene sequence, e.g., encoding granulocyte macrophage colony stimulating factor (GM-CSF). Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein. Hence, the terms “adenovirus vector” or “adenoviral vector” as used herein include adenovirus or adenoviral particles.

The term “gene transfer vehicle,” as used herein, means any construct which is capable of delivering a polynucleotide (DNA or RNA) sequence to a cell. Such gene transfer vehicles include, but are not limited to, viruses, such as adenoviruses, retroviruses, adeno-associated virus, Herpes viruses, plasmids, proteoliposomes which encapsulate a polynucleotide sequence to be transferred into a cell, and “synthetic viruses” and “synthetic vectors” which include a polynucleotide which is enclosed within a fusogenic polymer layer, or within an inner fusogenic polymer layer and an outer hydrophilic polymer layer.

The term as used herein “replication-competent” as used herein relative to the adenoviral vectors of the invention means the adenoviral vectors and particles of the invention preferentially replicate in certain types of cells or tissues but to a lesser degree or not at all in other types. In one embodiment of the invention, the adenoviral vector and/or particle selectively replicates in tumor cells and or abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700 and PCT publications WO 95/19434, WO 98/39465, WO 98/39467, WO 98/39466, WO 99/06576, WO 98/39464, and WO 00/15820. Such viruses may be referred to as “oncolytic viruses” or “oncolytic vectors” and may be considered to be “cytolytic” or “cytopathic” and to effect “selective cytolysis” of target cells.

The term “replication defective” as used herein relative to a viral vector of the invention means the vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

The terms “chimeric fiber protein” and “modified fiber protein” refers to an adenovirus fiber protein comprising a non-native amino acid sequence, in addition to or in place of a portion of a native fiber amino acid sequence. The non-native amino acid sequence may be from an adenoviral fiber protein of a different serotype. The non-native amino acid sequence may be any suitable length (e.g. 3 to about 200 amino acids). An exemplary “chimeric fiber protein” or “modified fiber protein” has a fiber shaft derived from one adenoviral serotype and a fiber head derived from a different adenoviral serotype.

The term “gene essential for replication” refers to a nucleotide sequence whose transcription is required for a viral vector to replicate in a target cell. For example, in an adenoviral vector of the invention, a gene essential for replication may be selected from the group consisting of the E1a, E1b, E2a, E2b, and E4 genes.

As used herein, a “packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

The terms “heterologous DNA” and “heterologous RNA” refer to nucleotides that are not endogenous (native) to the cell or part of the genome in which they are present. Generally heterologous DNA or RNA is added to a cell by transduction, infection, transfection, transformation or the like, as further described below. Such nucleotides generally include at least one coding sequence, but the coding sequence need not be expressed. The term “heterologous DNA” may refer to a “heterologous coding sequence” or a “transgene”.

As used herein, the terms “protein” and “polypeptide” may be used interchangeably and typically refer to “proteins” and “polypeptides” of interest that are expressed using the self processing cleavage site-containing vectors of the present invention. Such “proteins” and “polypeptides” may be any protein or polypeptide useful for research, diagnostic or therapeutic purposes, as further described below.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “native” refers to a gene or protein that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

In the context of the present invention, the term “isolated” refers to a nucleic acid molecule, polypeptide, virus, or cell that, by the hand of man, exists apart from its native environment and is therefore not a product of nature. An isolated nucleic acid molecule or polypeptide may exist in a purified form or may exist in a non-native environment such as, for example, a recombinant host cell. An isolated virus or cell may exist in a purified form, such as in a cell culture, or may exist in a non-native environment such as, for example, a recombinant or xenogeneic organism.

The term “operably linked” as used herein relative to a recombinant DNA construct or vector means nucleotide components of the recombinant DNA construct or vector pare functionally related to one another for operative control of a selected coding sequence. Generally, “operably linked” DNA sequences are contiguous, and, in the case of a secretory leader, contiguous and in reading frame. However, enhancers do not have to be contiguous.

As used herein, the term “gene” or “coding sequence” means the nucleotide polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “promoter” is a DNA sequence that directs the binding of RNA polymerase and thereby promotes RNA synthesis, i.e., a minimal sequence sufficient to direct transcription. Promoters and corresponding protein or polypeptide expression may be cell-type specific, tissue-specific, or species specific. Also included in the nucleic acid constructs or vectors of the invention are enhancer sequences which may or may not be contiguous with the promoter sequence. Enhancer sequences influence promoter-dependent gene expression and may be located in the 5′ or 3′ regions of the native gene.

“Enhancers” are cis-acting elements that stimulate or inhibit transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “regulatable promoter” is any promoter whose activity is affected by a cis or trans acting factor (e.g., an inducible promoter, such as an external signal or agent).

A “constitutive promoter” is any promoter that directs RNA production in many or all tissue/cell types at most times, e.g., the human CMV immediate early enhancer/promoter region which promotes constitutive expression of cloned DNA inserts in mammalian cells.

The term “E2F promoter” as used herein refers to a native E2F promoter and functional fragments, mutations and derivatives thereof. The E2F promoter does not have to be the full-length or wild type promoter. One skilled in the art knows how to derive fragments from an E2F promoter and test them for the desired selectivity. An E2F promoter fragment of the present invention has promoter activity selective for tumor cells, i.e. drives tumor selective expression of an operatively linked coding sequence. The term “tumor selective promoter activity” as used herein means that the promoter activity of a promoter fragment of the present invention in tumor cells is higher than in non-tumor cell types.

The term “telomerase promoter” or “TERT promoter” as used herein refers to a native TERT promoter and functional fragments, mutations and derivatives thereof. The TERT promoter does not have to be the full-length or wild type promoter. One skilled in the art knows how to derive fragments from a TERT promoter and test them for the desired selectivity. A TERT promoter fragment of the present invention has promoter activity selective for tumor cells, i.e. drives tumor selective expression of an operatively linked coding sequence. In one embodiment, the TERT promoter of the invention is a mammalian TERT promoter. In another embodiment, the mammalian TERT promoter is a human TERT promoter.

In one embodiment, an E2F promoter according to the present invention has a full-length complement that hybridizes to the sequence shown in SEQ ID NO:1 under stringent conditions. In another embodiment, the TERT promoter according to the present invention has a full-length complement that hybridizes to the sequence shown in SEQ ID NO:2 under stringent conditions. The phrase “hybridizing to” refers to the binding, duplexing, or hybridizing of a molecule to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays” Elsevier, New York. Generally, highly stringent hybridization and wash conditions are selected to be about 5° C. to 20° C. (preferably 5° C.) lower than the thermal melting point (Tₘ) for the specific sequence at a defined ionic strength and pH. Typically, under highly stringent conditions a probe will hybridize to its target subsequence, but to no other unrelated sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

The term “homologous” as used herein with reference to a nucleic acid molecule refers to a nucleic acid sequence naturally associated with a host virus or cell. The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

The terms “transcriptional regulatory protein”, “transcriptional regulatory factor” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to a DNA response element.

A “termination signal sequence” within the meaning of the invention may be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation.

As used herein, the terms “cancer”, “cancer cells”, “neoplastic cells”, “neoplasia”, “tumor”, and “tumor cells” (used interchangeably) refer to cells that exhibit relatively autonomous growth, so that they exhibit an aberrant growth phenotype or aberrant cell status characterized by a significant loss of control of cell proliferation. Neoplastic cells can be malignant or benign. It follows that cancer cells are considered to have an aberrant cell status.

Modified Adenoviruses

In accordance with an aspect of the present invention, there is provided a method of transferring at least one heterologous DNA sequence into cells. The method comprises transducing the cells with a modified adenovirus comprising the at least one heterologous DNA sequence. The adenovirus, prior to modification, is of a first serotype. In the modified adenovirus, at least a portion of the fiber of the adenovirus is removed and replaced with at least a portion of the fiber of an adenovirus of a second serotype. The cells include a receptor which binds to the at least a portion of the fiber of the adenovirus of the second serotype. Transfer of the at least one heterologous DNA sequence into said cells is effected through binding of the modified adenovirus to the cells.

As stated hereinabove, the adenovirus fiber protein includes a head region, a shaft region, and a tail region. In one embodiment, at least a part of the head region of the fiber of the adenovirus of the first serotype is removed and replaced with at least a part of the head region of the adenovirus of the second serotype. In a preferred embodiment, all of the head region of the fiber of the adenovirus of the first serotype is removed and replaced with the head region of the fiber of the adenovirus of the second serotype.

In one embodiment, the first and second serotypes of the adenoviruses are from different subgenera. In general, the human adenoviruses are divided into Subgenera A through F. Such subgenera are described further in Bailey, et al., Virology, Vol. 205, pgs. 438-452 (1994), the contents of which are herein incorporated by reference. Subgenus A includes Adenovirus 12, Adenovirus 18 and Adenovirus 31. Subgenus B includes Adenovirus 3, Adenovirus 7, Adenovirus 14, and Adenovirus 35. Subgenus C includes Adenovirus 1, Adenovirus 2, Adenovirus 5, and Adenovirus 6. Subgenus D includes Adenovirus 9, Adenovirus 10, Adenovirus 15, and Adenovirus 19. Subgenus E includes Adenovirus 4. Subgenus F includes Adenovirus 40 and Adenovirus 41. In one embodiment, the adenovirus of the first serotype is an Adenovirus of a serotype within Subgenus C, and the adenovirus of the second serotype is an adenovirus of a serotype within a subgenus selected from the group consisting of Subgenera A, B, D, E, and F. In another embodiment, the adenovirus of the second serotype is an adenovirus of a serotype within Subgenus B. In yet another embodiment, the adenovirus of the first serotype is Adenovirus 5, and the adenovirus of the second serotype is Adenovirus 3. In one example of this embodiment, amino acid residues 404 to 581 of the fiber (i.e., the fiber head region) of Adenovirus 5 are removed and replaced with amino acid residues 136 to 319 of the fiber (i.e., the fiber head region) of Adenovirus 3. The DNA encoding the fiber protein of Adenovirus 5 is registered as Genbank Accession No. M18369 (incorporated herein by reference), and the DNA encoding the fiber protein of Adenovirus 3 is registered as GenBank Accession No. M12411 (incorporated herein by reference).

Exemplary Ad3 fiber nucleotide and amino acid sequences are provided herein as SEQ ID NOs: 19 and 20, respectively (expressly incorporated herein by reference). Nucleotides 205-1209 of GenBank Accession No. X01998.1 are presented as SEQ ID NO:19. The 319 amino acid sequence for the Ad3 fiber protein from GenBank Accession No. ERADF3 is presented as SEQ ID NO:20 (Signas, C et al., J. Virol. 53 (2), 672-678, 1985; expressly incorporated herein by reference).

Nucleotides 1 to 1746 of the nucleotide sequence presented as SEQ ID NO: 15, which encodes an Ad5 fiber protein has 99% sequence identity to nucleotides 476 to 2221 of the adenovirus type 5 fiber gene sequence in GenBank Accession No M18369 (Chroboczek, J. and Jacrot B., Virology 161 (2), 549-554, 1987) and 99% sequence identity to nucleotides 31037 to 22782 of the human adenovirus C serotype 5 sequence in GenBank Accession No. AY339865.

Amino acids 1 to 581 of the amino acid sequence for the Ad 5 fiber presented as SEQ ID NO:16 has 94% sequence identity to amino acids 1 to 581 of GenBank Accession No. ERADF5, a human adenovirus 5 fiber protein sequence.

In yet another embodiment, the adenovirus of the first serotype is Adenovirus 5, and the adenovirus of the second serotype is Adenovirus 35. Thus, in such embodiment, amino acid residues 404 to 581 of the fiber (i.e., the fiber head region) of Adenovirus 5 are removed and replaced with amino acid residues 137 to 323 of the fiber (i.e., the fiber head region) of Adenovirus 35 (SEQ ID NO:14). As set forth above, the nucleotide sequence encoding the fiber protein of Adenovirus 5 is registered as Genbank Accession No. M18369.

Nucleotides 1 to 966 of the nucleotide sequence presented herein as SEQ ID NO: 13, the nucleotide sequence of the ORF encoding the Ad35 fiber protein has 100% sequence identity to nucleotides 1 to 966 of GenBank Accession No. HAU10272, a human adenovirus type 35p fiber coding sequence (expressly incorporated herein by reference).

An exemplary Ad35 fiber amino acid sequence is provided herein as SEQ ID NO: 14. A further example of a human Ad35 fiber amino acid sequence published prior to the priority filing date of the instant application (GenBank Accession No. AAA75331; Basler, C. et al., Gene 170:249-254, 1996), expressly incorporated herein by reference and presented as SEQ ID NO:21.

Cells which may be transduced with the modified adenoviruses described herein include cells which have a receptor that binds to the region of the fiber protein, and in particular the head region of the fiber protein, of the adenovirus of the second serotype. When the modified adenovirus is an adenovirus of the Adenovirus 5 serotype having a fiber head region of Adenovirus 3, the cells which may be transduced by such modified adenovirus include, but are not limited to, lung cells, including, but not limited to, lung epithelial cells and lung cancer cells; blood cells such as hematopoietic cells, including, but not limited to, monocytes and macrophages; lymphoma cells; leukemia cells, including acute myeloid leukemia cells and acute lymphocytic leukemia cells; smooth muscle cells, including, but not limited to, smooth muscle cells of blood vessels and of the digestive system; and tumor cells, including, but not limited to, head and neck cancer cells and neuroblastoma cells.

In one preferred embodiment, the modified adenovirus is a chimeric adenovirus wherein the majority of the fiber is from Adenovirus serotype 5 and the fiber head (knob) region is from Adenovirus 35. When using this Ad5/35 chimeric virus, the cells which may be transduced by such modified adenovirus include but are not limited to human head and neck cancer cell lines such as epidermoid carcinoma cells, squamous cell carcinoma (SQCC) cells, tongue SQCC cells, pharyngeal carcinoma cells, nasal septum SQCC cells and skin malignant melanoma cells.

Such adenoviruses may be constructed from an adenoviral vector of a first serotype wherein DNA encoding at least a portion of the fiber is removed and replaced with DNA encoding at least a portion of the fiber of the adenovirus of a second serotype.

The adenovirus, in general, also includes at least one heterologous DNA sequence to be transferred into cells. The at least one DNA sequence is typically a heterologous DNA sequence, and in particular, a heterologous DNA sequence encoding a therapeutic agent or transgene. The term “therapeutic” is used in a generic sense and includes treating agents, prophylactic agents, and replacement agents.

DNA sequences encoding therapeutic agents include, but are not limited to, DNA sequences encoding tumor necrosis factor (TNF) genes, such as TNF-.alpha.; genes encoding interferons such as Interferon-.alpha., Interferon-.beta., and Interferon-gamma.; genes encoding interleukins such as IL-1, IL-1β, and Interleukins 2 through 14; genes encoding G-CSF, GM-CSF, TGF-α, TGF-β, and fibroblast growth factor; genes encoding ornithine transcarbamylase, or OTC; genes encoding adenosine deaminase, or ADA; genes which encode cellular growth factors, such as lymphokines, which are growth factors for lymphocytes; genes encoding epidermal growth factor (EGF), vascular endothelial growth factor (VEGF), and keratinocyte growth factor (KGF); genes encoding soluble CD4; Factor VIII; Factor IX; cytochrome b; glucocerebrosidase; T-cell receptors; the LDL receptor, ApoE, ApoC, ApoAI and other genes involved in cholesterol transport and metabolism; the alpha-1 antitrypsin (.alpha.1AT) gene; genes encoding co-stimulatory antigens, such as B7.1; genes encoding chemotactic agents, such as lymphotactin, the cystic fibrosis transmembrane conductance regulator (CFTR) genes; the insulin gene; the hypoxanthine phosphoribosyl transferase gene; negative selective markers or “suicide” genes, such as viral thymidine kinase genes, such as the Herpes Simplex Virus thymidine kinase gene, the cytomegalovirus virus thymidine kinase gene, and the varicella-zoster virus thymidine kinase gene; Fc receptors for antigen-binding domains of antibodies, antisense sequences which inhibit viral replication, such as antisense sequences which inhibit replication of hepatitis B or hepatitis non-A non-B virus; antisense c-myb oligonucleotides; and antioxidants such as, but not limited to, manganese superoxide dismutase (Mn—SOD), catalase, copper-zinc-superoxide dismutase (CuMn—SOD), extracellular superoxide dismutase (EC—SOD), and glutathione reductase; tissue plasminogen activator (tPA); urinary plasminogen activator (urokinase); hirudin; the phenylalanine hydroxylase gene; nitric oxide synthetase; vasoactive peptides; angiogenic peptides; the dopamine gene; the dystrophin gene; the .beta.-globin gene; the alpha.-globin gene; the HbA gene; protooncogenes such as the ras, src, and bcl genes; tumor-suppressor genes such as p53 and Rb; genes encoding anti-angiogenic factors, such as, for example, endothelial monocyte activating polypeptide-2 (EMAP-2); the heregulin-.alpha. protein gene, for treating breast, ovarian, gastric and endometrial cancers; cell cycle control agent genes, such as, for example, the p21 gene; antisense polynucleotides to the cyclin G1 and cyclin D1 genes; the endothelial nitric oxide synthetase (ENOS) gene; monoclonal antibodies specific to epitopes contained within the .beta.-chain of a T-cell antigen receptor; the multidrug resistance (MDR) gene; the dihydrofolate reductase (DHFR) gene; DNA sequences encoding ribozymes; antisense polynucleotides; genes encoding secretory peptides which act as competitive inhibitors of angiotensin converting enzyme, of vascular smooth muscle calcium channels, or of adrenergic receptors, and DNA sequences encoding enzymes which break down amyloid plaques within the central nervous system. It is to be understood, however, that the scope of the present invention is not to be limited to any particular therapeutic agent.

In a preferred embodiment, the therapeutic agent is a cytokine, preferably granulocyte macrophage colony stimulating factor (GM-CSF) and the adenoviral vector comprises a heterologous nucleotide sequence encoding GM-CSF.

The heterologous DNA sequence which encodes the therapeutic agent may be genomic DNA or may be a cDNA sequence. The DNA sequence also may be the native DNA sequence or an allelic variant thereof. The term “allelic variant” as used herein means that the allelic variant is an alternative form of the native DNA sequence which may have a substitution, deletion, or addition of one or more nucleotides, which does not alter substantially the function of the encoded protein or polypeptide or fragment or derivative thereof. In one embodiment, the heterologous DNA sequence may further include a leader sequence or portion thereof, a secretory signal or portion thereof and/or may further include a trailer sequence or portion thereof.

The heterologous DNA sequence which encodes a therapeutic agent is under the control of a suitable promoter. Suitable promoters which may be employed include, but are not limited to, adenoviral promoters, such as the adenoviral major late promoter or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAI promoter. It is to be understood, however, that the scope of the present invention is not to be limited to specific foreign genes or promoters. In one preferred aspect of the invention the therapeutic agent is expressed under operative control of an adenoviral promoter.

The adenoviral vector which is employed may, in one embodiment, be an adenoviral vector which includes essentially the complete adenoviral genome (Shenk et al., Curr. Top. Microbiol. Immunol., 111(3): 1-39 (1984). Alternatively, the adenoviral vector may be a modified adenoviral vector in which at least a portion of the adenoviral genome has been deleted.

In one embodiment, the vector is free of at least the one gene taken from the adenoviral E3 region.

An adenoviral vector of the invention is typically constructed first by generating, according to standard techniques, a shuttle plasmid which contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a multiple cloning site (which may be as herein described); a poly A signal; and a DNA segment which corresponds to a segment of the adenoviral genome. The vector also may contain a tripartite leader sequence. The DNA segment corresponding to the adenoviral genome serves as a substrate for homologous recombination with a modified or mutated adenovirus, and such sequence may encompass, for example, a segment of the adenovirus 5 genome no longer than from base 3329 to base 6246 of the genome. The plasmid may also include a selectable marker and an origin of replication. The origin of replication may be a bacterial origin of replication. Representative examples of such shuttle plasmids include pAvS6, which is described in published PCT Application Nos. WO94/23582, published Oct. 27, 1994, and WO95/09654, published Apr. 13, 1995 and in U.S. Pat. No. 5,543,328, issued Aug. 6, 1996. The heterologous DNA sequence encoding a therapeutic agent then may be inserted into the multiple cloning site to produce a plasmid vector.

Other suitable promoters for regulating expression of an essential adenoviral gene include the human E2F promoter and the human telomerase promoter. Without being bound by theory, the selectivity of E2F-responsive promoters (hereinafter sometimes referred to as E2F promoters) is reported to be based on the derepression of the E2F promoter/transactivator in Rb-pathway defective tumor cells. In quiescent cells, E2F binds to the tumor suppressor protein pRB in ternary complexes. In its complexed form, E2F functions to repress transcriptional activity from promoters with E2F binding sites, including the E2F-1 promoter itself (Zwicker J, and Muller R., Prog. Cell Cycle Res 1995; 1:91-99). The E2F-1 promoter is transcriptionally inactive in resting cells. In normal cycling cells, pRB-E2F complexes are dissociated in a regulated fashion, allowing for controlled derepression of E2F and subsequent cell cycling (Dyson, N., Genes and Development 1998; 12:2245-2262).

In the majority of tumor types, the Rb cell cycle regulatory pathway is disrupted, suggesting that Rb-pathway deregulation is obligatory for tumorigenesis (Strauss M, Lukass J and Bartek J., Nat Med 1995; 12:1245-1246). One consequence of these mutations is the disruption of E2F-pRB binding and an increase in free E2F in tumor cells. Rb itself is mutated in some tumor types, and in other tumor types factors upstream of Rb are deregulated (Weinberg, R A. Cell 1995; 81:323-330). One effect of these Rb-pathway changes in tumors is the loss of pRB binding to E2F, and an apparent increase in free E2F in tumor cells. The abundance of free E2F in turn results in high-level expression of E2F responsive genes in tumor cells, including the E2F-1 gene. Accordingly, the term “Rb-pathway defective cells” may be functionally defined as cells which display an abundance of “free” E2F, as measured by gel mobility shift assay or by chromatin immunoprecipitation (Takahashi Y et al., Genes Dev. 2000 Apr. 1; 14(7):804-16). The E2F-1 promoter has been shown to up-regulate the expression of marker genes in an adenovirus vector in a rodent tumor model but not normal proliferating cells in vivo (Parr M J et al., Nature Med 1997; October; 3(10):1145-1149).

An E2F-responsive promoter has at least one E2F binding site. In one embodiment, the E2F-responsive promoter is a mammalian E2F promoter. In another embodiment, it is a human E2F promoter. For example, the E2F promoter may be the human E2F-1 promoter. Further, the human E2F-1 promoter may be, for example, a E2F-1 promoter having the sequence as described in SEQ ID NO:1. A number of examples of E2F promoters are known in the art (e.g. Parr et al. Nature Medicine 1997:3(10) 1145-1149, WO 02/067861, US20010053352 and WO 98/13508). E2F responsive promoters typically share common features such as Sp I and/or ATT7 sites in proximity to their E2F site(s), which are frequently located near the transcription start site, and lack of a recognizable TATA box. E2F-responsive promoters include E2F promoters such as the E2F-1 promoter, dihydrofolate reductase (DHFR) promoter, DNA polymerase A (DPA) promoter, c-myc promoter and the B-myb promoter. The E2F-1 promoter contains four E2F sites that act as transcriptional repressor elements in serum-starved cells. In one embodiment, an E2F-responsive promoter has at least two E2F sites. In another embodiment, an E2F promoter is operatively linked to the adenovirus E1a region. In a further embodiment, an E2F promoter is operatively linked to the adenovirus E1b region. In yet a further embodiment, an E2F promoter is operatively linked to the adenovirus E4 region.

In one embodiment of the invention, the recombinant viral vectors of the present invention selectively replicate in and lyse Rb-pathway defective cells. In one embodiment, the E2F promoter of the invention is a mammalian E2F promoter. In another embodiment, the mammalian E2F promoter is a human E2F promoter, for example a human E2F promoter which comprises or consists essentially of SEQ ID NO:1. Embodiments of the invention include adenoviral vectors comprising an E2F promoter wherein the E2F promoter comprises a nucleotide sequence selected from the group consisting of: (a) the sequence shown in SEQ ID NO:1; (b) a fragment of the sequence shown in SEQ ID NO: 1, wherein the fragment has tumor selective promoter activity; (c) a nucleotide sequence having at least 90, 91, 92, 93, 94, 95, 96, 97, 98, 99% or more % identity over its entire length to the sequence shown in SEQ ID NO: 1, wherein the nucleotide sequence has tumor selective promoter activity; and (d) a nucleotide sequence having a full-length complement that hybridizes under stringent conditions to the sequence shown in SEQ ID NO:1, wherein the nucleotide sequence has tumor selective promoter activity. In another embodiment of the invention, the E2F promoter comprises nucleotides 7 to 270 of SEQ ID NO:1. In another embodiment of the invention, the E2F promoter comprises nucleotides 7 to 270 of SEQ ID NO:1, wherein nucleotide 75 of SEQ ID NO:1 is a T instead of a C.

In other embodiments, a E2F promoter according to the present invention has at least 80, 85, 87, 89, 90, 91, 92, 93, 94, 95, 96, 97, 98, 99% or more sequence identity to the sequence shown in SEQ ID NO:1, when compared and aligned for maximum correspondence, as measured using one of the following sequence comparison algorithms or by visual inspection. In one embodiment, the given % sequence identity exists over a region of the sequences that is at least about 50 nucleotides in length. In another embodiment, the given % sequence identity exists over a region of at least about 100 nucleotides in length. In another embodiment, the given % sequence identity exists over a region of at least about 200 nucleotides in length. In another embodiment, the given % sequence identity exists over the entire length of the sequence.

The E2F-responsive promoter does not have to be the full-length or wild type promoter, but should have a tumor-selectivity of at least 3-fold, at least 5-fold, at least 10-fold, at least 20-fold, at least 30-fold, at least 50-fold, at least 100-fold or even at least 300-fold. Tumor-selectivity can be determined by a number of assays using known techniques, such as the techniques employed in WO 02/067861, Example 4, for example RT-PCR or a comparison of replication in selected cell types. The tumor-selectivity of the adenoviral vectors can also be quantified by E1A RNA levels, as further described in WO 02/067861, Example 4, and the E1A RNA levels obtained in H460 (ATCC, Cat. # HTB-177) cells can be compared to those in PERC (Clonetics Cat. #CC2555) cells in order to determine tumor-selectivity for the purposes of this invention. The relevant conditions of the experiment may vary, but typically follow those described in WO 02/067861.

Without being bound by theory, the understanding of selective TERT expression in cancer is based on the current knowledge that TERT is the rate-limiting catalytic subunit of telomerase, a multicomponent ribonucleoprotein enzyme that has also been shown to be active in ˜85% of human cancers but not normal somatic cells (Kilian A et al. Hum Mol Genet. 1997 November; 6(12):2011-9; Kim N W et al. Science. 1994 Dec. 23; 266(5193):2011-5; Shay J W et al. European Journal of Cancer 1997; 5, 787-791; Stewart S A et al. Semin Cancer Biol. 2000 December; 10(6):399-406). Cancer cells appear to require immortalization for tumorigenesis and telomerase activity is almost always necessary for immortalization (Kim N W et al. Science. 1994 Dec. 23; 266(5193):2011-5; Kiyono T et al. Nature 1998; 396:84). Thus, the majority of tumor cells have a disregulated telomerase pathway. Such tumor cells are specifically targeted by viruses of the invention utilizing a TERT promoter operatively linked to a gene and/or coding region essential for replication (e.g. E1a, E1b or E4).

The term TERT promoter as used herein refers to a full-length TERT promoter and functional fragments, mutations and derivatives thereof. The TERT promoter does not have to be a full-length or wild type promoter. One skilled in the art knows how to derive fragments from a TERT promoter and test them for the desired specificity. In one embodiment, a TERT promoter of the invention is a mammalian TERT promoter. In a further embodiment the mammalian TERT promoter, is a human TERT promoter (hTERT). In one embodiment of the invention, the TERT promoter comprises or consists essentially of SEQ ID NO:2, which is a 239 bp fragment of the hTERT promoter. In another embodiment of the invention, the TERT promoter comprises or consists essentially of SEQ ID NO:3, which is a 245 bp fragment of the hTERT promoter. In one embodiment, a TERT promoter is operatively linked to the adenovirus E1a region. In another embodiment, the TERT promoter is operatively linked to the adenovirus E1b region. In yet a further embodiment, the TERT promoter is operatively linked to the adenovirus E4 region.

Embodiments of the invention include adenoviral vectors comprising a TERT promoter wherein the TERT promoter comprises a nucleotide sequence selected from the group consisting of: (a) the sequence shown in SEQ ID NO:2; (b) a fragment of the sequence shown in SEQ ID NO:2, wherein the fragment has tumor selective promoter activity; (c) a nucleotide sequence having at least 90% identity over its entire length to the sequence shown in SEQ ID NO:2, wherein the nucleotide sequence has tumor selective promoter activity; and (f) a nucleotide sequence having a full-length complement that hybridizes under stringent conditions to the sequence shown in SEQ ID NO:2, wherein the nucleotide sequence has tumor selective promoter activity. Other examples of TERT promoters are known to those skilled in the art (e.g. WO 98/14593).

In other embodiments, an TERT promoter according to the present invention has at least In other embodiments, a E2F promoter according to the present invention has at least 80, 85, 87, 89, 90, 91, 92, 93, 94, 95, 96, 97, 98, 99% or more sequence identity to the sequence shown in SEQ ID NO:2 or SEQ ID NO:3, when compared and aligned for maximum correspondence, as measured using one of the following sequence comparison algorithms or by visual inspection. In one embodiment, the given % sequence identity exists over a region of the sequences that is at least about 50 nucleotides in length. In another embodiment, the given % sequence identity exists over a region of at least about 100 nucleotide. In another embodiment, the given % sequence identity exists over a region of at least about 200 nucleotides. In another embodiment, the given % sequence identity exists over the entire length of the sequence.

Upon formation of the adenoviral vectors hereinabove described, the genome of such a vector is modified such that DNA encoding at least a portion of the fiber protein is removed and replaced with DNA encoding at least a portion of the fiber protein an adenovirus having a serotype different from that of the adenovirus being modified. Such modification may be accomplished through genetic engineering techniques known to those skilled in the art.

Upon modification of the genome of the adenoviral vector, the vector is transfected into an appropriate cell line for the generation of infectious adenoviral particles wherein at least a portion of the fiber protein, in particular the head region has been changed to include a portion, and in particular the head region, of the fiber protein of an adenovirus having a serotype different from that of the adenovirus being modified.

Alternatively, a DNA sequence encoding a modified fiber may be placed into an adenoviral shuttle plasmid such as those hereinabove described. The shuttle plasmid also may include a heterologous DNA sequence encoding a therapeutic agent. The shuttle plasmid is transfected into an appropriate cell line for the generation of infectious viral particles, with an adenoviral genome wherein the DNA encoding the fiber protein is deleted.

In another alternative, a first shuttle plasmid includes a heterologous DNA sequence encoding the therapeutic agent, and a second shuttle plasmid includes a DNA sequence encoding the modified fiber. The first shuttle plasmid is transfected into an appropriate cell line for the generation of infectious viral particles including a heterologous DNA sequence encoding a therapeutic agent. The second shuttle plasmid, which includes the DNA sequence encoding the modified fiber, is transfected with the adenovirus including the heterologous DNA sequence encoding a therapeutic agent into an appropriate cell line to generate infectious viral particles including the modified fiber and heterologous therapeutic agent-encoding DNA sequence through homologous recombination.

In yet another alternative, the modified adenovirus is constructed by effecting homologous recombination between an adenoviral vector of the first serotype which includes a heterologous DNA sequence encoding a therapeutic agent, with a shuttle plasmid including a DNA sequence encoding a modified fiber.

The modified adenovirus may be employed to transduce cells in vivo, ex vivo, or in vitro. When administered in vivo, the adenoviruses of the present invention may be administered in an amount effective to provide a therapeutic effect in a host. In one embodiment, the modified adenovirus may be administered in an amount of from 1 plaque-forming unit to about 1014 plaque forming units, preferably from about 106 plaque forming units to about 1013 plaque forming units. The host may be a mammalian host, including human or non-human primate hosts.

The modified adenovirus may be administered in combination with a pharmaceutically acceptable carrier suitable for administration to a patient, such as, for example, a liquid carrier such as a saline solution, protamine sulfate (Elkins-Sinn, Inc., Cherry Hill, N.J.), or Polybrene (Sigma Chemical).

Cells which may be transduced with the modified adenovirus are those which include a receptor for the adenovirus of the second serotype, whereby the portion of the fiber of the adenovirus of the second serotype, in particular the head region, which is included in the modified adenovirus, is bound by the receptor for the adenovirus of the second serotype.

Ad5/Ad3 Chimeric Fiber Proteins

When, as in one embodiment, the adenovirus of the first serotype is Adenovirus 5, and such adenovirus has been modified such that at least a portion of the fiber, in particular the head region of Adenovirus 5, has been removed and replaced with at least a portion, in particular the head region of Adenovirus 3, cells which may be transduced include lung cells, including normal lung cells such as lung epithelial cells, lung fibroblasts, and lung cancer cells; blood cells, such as hematopoietic cells, including monocytes and macrophages; lymphoma cells; leukemia cells, including acute myeloid leukemia cells and acute lymphocytic leukemia cells; smooth muscle cells, including smooth muscle cells of blood vessels and of the digestive system; and tumor cells, including head and neck cancer cells, lung cancer cells, and neuroblastoma cells.

Thus, a modified adenovirus of the Adenovirus 5 serotype which includes a head portion of the fiber of Adenovirus 3 may be used to treat a disease or disorder of the lung (such as, for example, cystic fibrosis, lung surfactant protein deficiency states, or emphysema). The modified adenovirus may be administered, for example, by aerosolized inhalation or bronchoscopic installation, or via intranasal or intratracheal instillation. For example, the modified adenoviruses may be used to infect lung cells, and such modified adenoviruses may include the CFTR gene, which is useful in the treatment of cystic fibrosis. In another embodiment, the modified adenovirus may include a gene(s) encoding a lung surfactant protein, such as surfactant protein A (SP-A), surfactant protein B (SP-B), or surfactant protein C(SP-C), whereby the modified adenoviral vector is employed to treat lung surfactant protein deficiency states. In yet another embodiment, the modified adenovirus may include a gene encoding alpha.-1-antitrypsin, whereby the modified adenovirus may be employed in the treatment of emphysema caused by alpha.-1-antitrypsin deficiency.

In another embodiment, the modified adenoviruses may be used to infect hematopoietic stem cells of a cancer patient undergoing chemotherapy in order to protect such cells from adverse effects of chemotherapeutic agents. Such cells may be transduced with the modified adenovirus in vivo, or the cells may be obtained from a blood sample or bone marrow sample that is removed from the patient, transduced with the modified adenovirus ex vivo, and returned to the patient. For example, hematopoietic stem cells may be transduced in vivo or ex vivo with a modified adenovirus of the present invention which includes a multidrug resistance (MDR) gene or a dihydrofolate reductase (DHFR) gene. Such transduced hematopoietic stem cells become resistant to chemotherapeutic agents, and therefore such transduced hematopoietic stem cells can survive in cancer patients that are being treated with chemotherapeutic agents.

In yet another embodiment, the modified adenoviruses may be employed in the treatment of tumors, such as head and neck cancer, neuroblastoma, lung cancer, and lymphomas. For example, the modified adenovirus may include a negative selective marker, or “suicide” gene, such as the Herpes Simplex Virus thymidine kinase (TK) gene. The modified adenovirus may be employed in the treatment of the head and neck cancer or lung cancer, or neuroblastoma, or lymphoma, by administering the modified adenovirus to a patient, such as, for example, by direct injection of the modified adenovirus into the tumor or into the lymphoma, whereby the modified adenovirus transduces the tumor cells or lymphoma cells. Alternatively, when the modified adenovirus is employed to treat head and neck cancer or neuroblastoma, the modified adenovirus may be administered to the vasculature at a site proximate to the head and neck cancer or neuroblastoma, whereby the modified adenovirus travels to and transduces the head and neck cancer cells or neuroblastoma cells. After the tumor cells or lymphoma cells are transduced with the modified adenovirus, an interaction agent or prodrug, such as, for example, ganciclovir, is administered to the patient, whereby the transduced tumor cells are killed.

In a further embodiment, the modified adenoviruses may be employed in the treatment of leukemias, including acute myeloid leukemia and acute lymphocytic leukemia. For example, the modified adenovirus may include a negative selective marker, or “suicide” gene, such as hereinabove described. The modified adenovirus may be administered intravascularly, or the modified adenovirus may be administered to the bone marrow, whereby the modified adenovirus transduces the leukemia cells. After the leukemia cells are transduced with the modified adenovirus, an interaction agent or prodrug is administered to the patient, whereby the transduced leukemia cells are killed.

In an alternative embodiment, leukemias, including acute myeloid leukemia and acute lymphocytic leukemia, or neuroblastoma, may be treated with a modified adenovirus including a DNA sequence encoding a polypeptide which elicits an immune response against the leukemia cells or neuroblastoma cells. Such polypeptides include, but are not limited to, immunostimulatory cyctokines such as Interleukin-2, co-stimulatory antigens, such as B7.1; and chemotactic agents, such as lymphotactin. When employed to treat leukemia, the modified adenovirus may be administered intravascularly, or may be administered to the bone marrow, whereby the modified adenovirus transduces the leukemia cells. When employed to treat neuroblastoma, the modified adenovirus may be administered directly to the neuroblastoma, and/or may be administered intravascularly, whereby the modified adenovirus transduces the neuroblastoma cells.

The transduced leukemia cells or the transduced neuroblastoma cells then express the polypeptide which elicits an immune response against the leukemia cells or the neuroblastoma cells, thereby inhibiting, preventing, or destroying the growth of the leukemia cells or neuroblastoma cells.

In yet another embodiment, the modified adenovirus may be employed to prevent or treat restenosis or prevent or treat vascular lesions after an invasive vascular procedure. The term “invasive vascular procedure,” as used herein, means any procedure that involves repair, removal, replacement, and/or redirection (e.g., bypass or shunt) of a portion of the vascular system, including, but not limited to arteries and veins. Such procedures include, but are not limited to, angioplasty, vascular grafts such as arterial grafts, removals of blood clots, removals of portions of arteries or veins, and coronary bypass surgery. For example, the modified adenovirus may include a heterologous DNA sequence encoding a therapeutic agent, such as cell cycle control agents, such as, for example, p21; hirudin; endothelial nitric oxide synthetase; or antagonists to cyclin G1 or cyclin D1, such as antibodies which recognize an epitope of cyclin G1 as cyclin D1. Alternatively, the modified adenovirus may include an antisense polynucleotide to the cyclin G1 or cyclin D1 gene, or in another alternative, the modified adenovirus may include a negative selective marker or “suicide” gene as hereinabove described. The modified adenovirus then is administered intravascularly, at a site proximate to the vascular lesion, or to the invasive vascular procedure, whereby the modified adenovirus transduces smooth muscle cells of the vasculature. The transduced cells then express the therapeutic agent, thereby treating or preventing restenosis or vascular lesions. Such restenosis or vascular lesions include, but are not limited to, restenosis or lesions of the coronary, carotid, femoral, or renal arteries, and renal dialysis fistulas.

In one embodiment, when the restenosis or vascular lesion is associated with proliferation of smooth muscle cells of the vasculature, the modified adenovirus may include a gene encoding a negative selective marker, or “suicide” gene as hereinabove described. Upon transduction of the smooth muscle cells with the modified adenovirus, an interaction agent or prodrug as hereinabove described is administered to the patient, thereby killing the transduced smooth muscle cells at the site of the restenosis or vascular lesion, and thereby treating the restenosis or vascular lesion.

Ad5/Ad35 Chimeric Fiber Proteins

In another embodiment, the adenovirus is Adenovirus 5, and is modified such that at least a portion of the fiber, in particular the head region of Adenovirus 5, has been removed and replaced with at least a portion, in particular the head region of Adenovirus 35.

Thus, a modified adenovirus of the Adenovirus 5 serotype which includes a head region of the fiber of Adenovirus 35 may be used to transduce cells including lung cells, including epidermoid cells, tongue cells, pharyngeal cells, nasal septum cells, skin cells and tumor cells, including head and neck cancer cells and melanoma cells and for use in treating a disease or disorder of the tongue, pharynx, nasal septum or skin, such as epidermoid carcinoma, squamous cell carcinoma (SQCC), tongue SQCC, pharyngeal carcinoma, nasal septum SQCC and malignant melanoma.

In addition, GenBank AAA75331 discloses the sequence of an Ad35 fiber. This is an exemplary sequence and a number of genomic variants exist (Flomenberg et al., J. Infec. Dis., 155(6) 1127-1134 (1987)). In practicing the present invention, the portion of the adenoviral protein derived from Ad35 head region may be from any Ad35 genomic variant.

Given the Ad5 and Ad35 sequence information known in the art and the instruction provided herein, one skilled in the art can combine an Adenovirus of serotype 5 (i.e. the fiber shaft and tail regions) with the fiber head (also termed the “knob”) of an adenovirus of serotype 3 or 35 in order to generate a vector which exhibits enhanced transduction of tumor cells, e.g., primary tumor cells and tumor cell lines. The details as to how to generate adenovirus with a chimeric fiber protein will be readily apparent to those of skill in the art given the disclosure provided herein and the detailed sequence information available as of the priority filing date of the instant application.

In one embodiment, the chimeric fiber protein comprises the complete adenovirus serotype 5 (Ad5) fiber shaft (amino acids 47 to 399 of SEQ ID NO:16). In another embodiment, the chimeric fiber protein comprises the head region from an adenovirus serotype 35 fiber protein (amino acids 137 to 323 of SEQ ID NO:14 or SEQ ID NO:21). In other embodiments, the chimeric fiber protein comprises the complete adenovirus serotype 5 (Ad5) fiber shaft (amino acids 47 to 399 of SEQ ID NO:16) and the head region from an adenovirus serotype 35 fiber protein (amino acids 137 to 323 of SEQ ID NO:14 or SEQ ID NO:21).

It will be understood by those of skill in the art that the exact sequence locations where the adenovirus serotype 5 (Ad5) fiber shaft is joined to a head or knob sequence taken from adenovirus serotype 3 (Ad3) or 35 (Ad35) may vary, so long as the resulting chimeric fiber protein functions. An adenovirus having a modified or chimeric fiber protein according to the present invention has a functional fiber protein if the adenovirus can enter a target cell and replicate.

In one embodiment, the Ad5 or Ad2 shaft region retains the KKTK sequence (SEQ ID NO:9). In an alternative embodiment the KKTK sequence in the native shaft sequence is deleted or mutated. In one embodiment, the Ad5 shaft retains the KLGTGLSFD sequence (SEQ ID NO:10) (Wu et al. J. Virol. 2003 July; 77(13):7225-35), In one embodiment, the Ad5 shaft retains the GNLTSQNVTTVSPPLKKTK (SEQ ID NO:11) comprising the third repeat region of the shaft with flexibility domain. In an alternative embodiment, Ad35 shaft contains the third repeat of the shaft (GTLQENIRATAPITKNN; (SEQ ID NO: 11), which lacks the sequence responsible for flexibility of the fiber.

The chimeric Ad5/Ad35 fiber proteins may include further modifications including, but not limited to modifications that decrease binding of the viral vector particle to a particular cell type or more than one cell type, enhance the binding of the viral vector particle to a particular cell type or more than one cell type and/or reduce the immune response to the adenoviral vector in an animal. Examples of these modifications include, but are not limited to those described in U.S. application Ser. No. 10/403,337, WO 98/07877, WO 01/92299, WO 2003/62400 and U.S. Pat. Nos. 5,962,311, 6,153,435, 6,455,314 and Wu et al. (J Virol. 2003 Jul. 1; 77(13):7225-7235). A non-native ligand may be included in the HI loop or at the carboxyl end of the chimeric fiber protein

In a preferred embodiment, the Ad5/Ad35 chimeric fiber vectors encodes a therapeutic agent, preferably a cytokine such as GM-CSF.

Gene Delivery Vehicles

In another embodiment, the modified adenovirus, which includes a heterologous DNA sequence encoding a therapeutic agent, may be administered to an animal in order to use such animal as a model for studying a disease or disorder and the treatment thereof. For example, a modified adenovirus, in accordance with the present invention, containing a DNA sequence encoding a therapeutic agent may be given to an animal which is deficient in such therapeutic agent. Subsequent to the administration of such modified adenovirus containing the DNA sequence encoding the therapeutic agent, the animal is evaluated for expression of such therapeutic agent. From the results of such a study, one then may determine how such adenoviruses may be administered to human patients for the treatment of the disease or disorder associated with the deficiency of the therapeutic agent.

It is also contemplated within the scope of the present invention that at least a portion, preferably at least a portion of the head region, and more preferably the entire head region, of the fiber of an adenovirus of a desired serotype may be incorporated into a gene delivery or gene transfer vehicle other than an adenovirus. Such gene delivery or gene transfer vehicles include, but are not limited to, viral vectors such as retroviral vectors, adeno-associated virus vectors, and Herpes virus vectors, such as Herpes Simplex Virus vectors; and non-viral gene delivery systems, including plasmid vectors, proteoliposomes encapsulating genetic material, “synthetic viruses,” and “synthetic vectors.”

When a viral vector is employed, the viral surface protein, such as a retroviral envelope, an adeno-associated virus naked protein coat, or a Herpes Virus envelope, is modified to include at least a portion, preferably at least a portion of the head region, and more preferably the entire head region, of an adenovirus of a desired serotype, whereby the viral vector may be employed to transduce cells having a receptor which binds to the head region of the fiber of the adenovirus of the desired serotype. For example, the viral vector, which includes a polynucleotide (DNA or RNA) sequence to be transferred into a cell, may have a viral surface protein which has been modified to include the head region of the fiber of Adenovirus 3. Such viral vectors may be constructed in accordance with genetic engineering techniques known to those skilled in the art. The viral vectors then may be employed to transduce cells, such as those hereinabove described, which include a receptor which binds to the head region of the fiber of Adenovirus 3, to treat diseases or disorders such as those hereinabove described.

In another embodiment, the gene transfer vehicle may be a plasmid, to which is linked at least a portion, preferably at least a portion of the head region, and more preferably the entire head region, of the fiber of an adenovirus of a desired serotype. The at least a portion of the fiber of the adenovirus of a desired serotype may be bound directly to the plasmid vector including a polynucleotide to be transferred into a cell, or the at least a portion of the fiber of the adenovirus of a desired serotype may be attached to the plasmid vector by means of a linker moiety, such as, for example, linear and branched cationic polymers, such as, polyethyleneimine, or a polylysine conjugate, or a dendrimer polymer. The plasmid vector then is employed to transduce cells having a receptor which binds to the head region of the fiber of the adenovirus of the desired serotype. For example, a plasmid vector may be attached, either through direct binding or through a linker moiety, to the head portion of the fiber of Adenovirus 3. The plasmid vector then may be employed to transduce cells having a receptor which binds to the head region of the fiber of Adenovirus 3, as hereinabove described.

In another embodiment, a polynucleotide which is to be transferred into a cell may be encapsulated within a proteoliposome which includes at least a portion, preferably at least a portion of the head region, and more preferably the entire head region, of the fiber of an adenovirus of a desired serotype. The polynucleotide to be transferred to a cell may be a naked polynucleotide sequence or may be contained in an appropriate expression vehicle, such as a plasmid vector. The proteoliposome may be formed by means known to those skilled in the art. The proteoliposome, which encapsulates the polynucleotide sequence to be transferred to a cell, is employed in transferring the polynucleotide to cells having a receptor which binds to the head region of the fiber of the adenovirus of a desired serotype. For example, the proteoliposome may include, in the wall of the proteoliposome, the head region of the fiber of Adenovirus 3, and such proteoliposome may be employed in contacting cells, such as those hereinabove described, which include a receptor which binds to the head region of the fiber of Adenovirus 3. Upon binding of the proteoliposome to the cell, the polynucleotide contained in the liposome is transferred to the cell.

In yet another embodiment, a polynucleotide which is to be transferred into the cell may be part of a “synthetic virus.” In such a “synthetic virus,” the polynucleotide is enclosed within an inner fusogenic layer of a pH sensitive membrane destabilizing polymer. The “synthetic virus” also includes an outer layer of a cleavable hydrophilic polymer. The at least a portion, preferably at least a portion of the head region, and more preferably the entire head region, of the fiber of an adenovirus of a desired serotype, is bound to the outer layer of the cleavable hydrophilic polymer. The polynucleotide to be transferred to a cell may be a naked polynucleotide sequence or may be contained in an appropriate expression vehicle as hereinabove described. The “synthetic virus” is employed in transferring the polynucleotide to cells having a receptor which binds to the head region of the fiber of the adenovirus of a desired serotype. For example, the “synthetic virus” may include the head portion of the fiber of Adenovirus 3, which is bound to the cleavable hydrophilic polymer. The “synthetic virus” is employed in contacting cells which include a receptor which binds to the head region of the fiber of Adenovirus 3. Upon binding of the “synthetic virus” to the cell, the polynucleotide contained in the “synthetic virus” is transferred to the cell.

In a further embodiment, a polynucleotide which is to be transferred into a cell may be part of a “synthetic vector”, wherein the polynucleotide is enclosed within a fusogenic layer of a fusogenic pH sensitive membrane destabilizing polymer. The at least a portion, preferably at least a portion of the head region, and more preferably the entire head region, of the fiber of an adenovirus of a desired serotype, is bound to the fusogenic pH sensitive membrane destabilizing polymer. Such a “synthetic vector” is useful especially for transferring polynucleotides to cells ex vivo or in vitro. For example, the “synthetic vector” may include the head portion of the fiber of Adenovirus 3, which is bound to the fusogenic pH sensitive membrane destabilizing polymer. The “synthetic vector” is employed in contacting cells which includes a receptor which binds to the head region of the fiber of Adenovirus 3. Upon binding of the “synthetic vector” to the cell, the polynucleotide contained in the “synthetic vector” is transferred to the cell.

In accordance with yet another aspect of the present invention, there is provided an adenoviral vector of the Adenovirus 3 or 35 serotype which includes at least one heterologous DNA sequence. The at least one heterologous DNA sequence may be selected from those hereinabove described. Such adenoviral vectors may be employed in transducing cells, such as those hereinabove described, either in vivo, ex vivo, or in vitro, which include a receptor which binds to the head region of the Adenovirus 3. The vectors may be administered in dosages such as those hereinabove described. The vectors may be administered in combination with a pharmaceutically acceptable carrier, such as those hereinabove described. Thus, such vectors may be employed to treat diseases or disorders such as those hereinabove described. It is to be understood, however, that the scope of this aspect of the present invention is not to be limited to the transduction of any particular cell type or the treatment of any particular disease or disorder.

Thus, in accordance with another aspect of the present invention, there is provided a method of transferring at least one polynucleotide into cells by contacting the cells with a gene transfer vehicle which includes at least a portion, preferably at least a portion of the head region, and more preferably the entire head region, of the fiber of Adenovirus 3. The cells include a receptor which binds to the at least a portion of the fiber of Adenovirus 3. Transfer of the at least one polynucleotide sequence into cells is effected through binding of the gene transfer vehicle to the cells. Such gene transfer vehicles include, but are not limited to, adenoviruses; retroviruses; adeno-associated virus; Herpes viruses such as Herpes Simplex Virus; plasmid vectors bound to the at least a portion, preferably the head region, of the fiber of Adenovirus 3; and proteoliposomes encapsulating at least one polynucleotide to be transferred into cells. The at least one polynucleotide may encode at least one therapeutic agent such as those hereinabove described.

### EXAMPLES

The invention now will be described with respect to the following examples; however, the scope of the present invention is not intended to be limited thereby.

### Example 1

Recombinant Ad5/Ad3 fiber plasmid. A shuttle plasmid was constructed for homologous recombination at the right hand end of Adenovirus 5 based adenoviral vectors. This shuttle plasmid, referred to as prepac, contains the last 8886 bp from 25171 bp to 34057 bp of the Ad d1327 (Thimmapaya, Cell, Vol. 31, pg. 543 (1983)) genome cloned into pBluescript SK II(+) (Stratagene) and was kindly supplied by Dr. Soumitra Roy, Genetic Therapy, Inc., Gaithersburg, Md. The wild type, Adenovirus 5 fiber cDNA contained within prepac was replaced with the 5TS3Ha cDNA using PCR gene overlap extension, as described in Horton, et al., Biotechniques, Vol. 8, pgs. 528-535 (1990). The 5TS3H contains the Adenovirus 5 fiber tail and shaft domains (5TS; amino acids 1 to 403) fused with the Adenovirus 3 fiber head region (3H, amino acids 136 to 319) as described in Stevenson, et al., J. Virol., Vol. 69, pgs. 2850-2857 (1995). The 5TS3Ha cDNA was modified to contain native 3′ downstream sequences of the wildtype 5F cDNA. In addition, the last two codons of the Adenovirus 3 fiber head domain, GAC TGA were mutated to correspond to the wild type, 5F codon sequence, GAA TAA to maintain the Adenovirus 5 fiber stop codon and polyadenylation signal. The Adenovirus 5 fiber 3′ downstream sequences were added onto the 5TS3Ha cDNA using the pgem5TS3H plasmid (Stevenson, 1995) as template and the following primers: P1:5′-CATCTGCAGCATGAAGCGCGCAAGACCGTCTGAAGATA-3′ (scs4; SEQ ID NO:5) and P2:5′-CGTTGAAACATAACACAAACGAITCTTTATTCATCTTCTCTAATATAGGAAAAGGTAAk-3′ (scs 80; SEQ ID NO: 6). Overlapping homologous sequences were added onto prepac using the following primers: P3,5′-TTACCTTTTCCTATATTAGAGAAGATGAATAAAGAATCGTTTGTGTTATGTTTCAACG-3′ (scs 79; SEQ ID NO:7) and P4,5′-AGACAAGCTTGCATGCCTGCAGGACGGAGC-3′ (scs81; SEQ ID NO:8). Amplified products of the expected size were obtained and were gel purified. A second PCR reaction was carried out using the end primers, P1 and P4 to join the two fragments together. The DNA fragment generated in the second PCR reaction contained the 5TS3Ha cDNA with the last two codons mutated to the wildtype 5F sequence and the appropriate 3′ downstream prepac sequences. The 5TS3Ha PCR fragment was digested with NdeI and Sse8387 and was cloned directly into prepac to create the fiber shuttle plasmid, prep5TS3Ha.

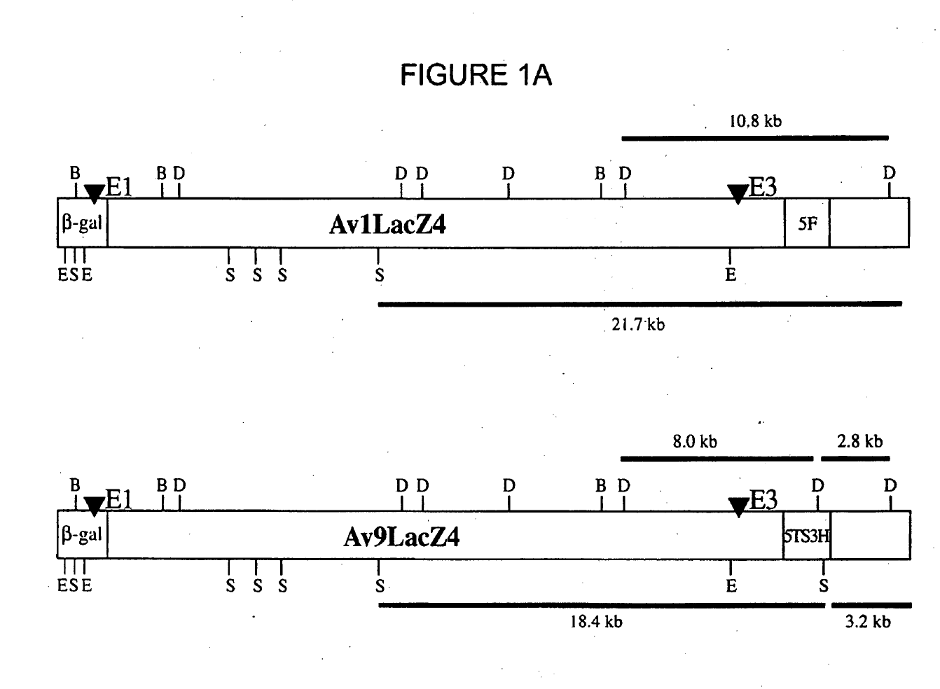

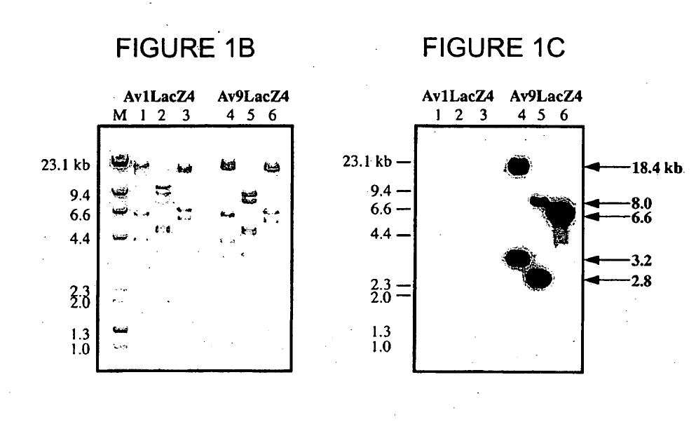

Generation of recombinant Ad5/Ad3 adenoviruses. The modified STS3Ha fiber cDNA was incorporated into the genome of Av1LacZ4, an E1 and E3-deleted adenoviral vector encoding β-galactosidase, and described in PCT Application No. WO95/09654, published Apr. 13, 1995, by homologous recombination between Av1LacZ4 and the prep5TS3Ha fiber shuttle plasmid to generate the chimeric fiber adenoviral vector referred to as Av9LacZ4. Human embryonic kidney 293 cells (ATCC CCL-1573) were obtained from the American Type Culture Collection (Rockville, Md.) and cultured in IMEM containing 10% heat inactivated FBS (HIFBS). Co-transfections of 293 cells were carried out with 10 μg of NotI-digested prep5TS3Ha and 1.5 μg of SrfI-digested Av1LacZ4 genomic DNA using a calcium phosphate mammalian transfection system (Promega Corporation, Madison, Wis.). The 293 cells were incubated with the calcium phosphate, DNA precipitate at 37° C. for 24 hours. The precipitate was removed and the monolayers were washed once with phosphate buffered saline (PBS). The transfected 293 cell monolayers were overlayered with 1% Sea Plaque agarose in MEM supplemented with 7.5% HIFBS, 2 mM glutamine, 50 units/ml penicillin, 50 μg/ml streptomycin sulfate, and 1% amphotericin B. Adenoviral plaques were isolated after approximately 14 days. Individual plaques were expanded, genomic DNA was isolated and screened for the presence of the chimeric fiber, 5TS3Ha cDNA using ScaI restriction enzyme digestion and confirmed by Southern blot analysis using the Ad3 fiber head as probe. Positive plaques were subjected to two rounds of plaque purification to remove parental, Av1LacZ4 contamination. The Av9LacZ4 vector after two rounds of plaque purification was expanded and purified by conventional techniques using CsCl ultracentrifugation. The adenovirus titers (particles/ml) were determined spectrophotometrically (Halbert, et al., J. Virol., Vol. 56, pgs. 250-257 (1985); Weiden, et al., Proc. Nat. Acad. Sci., Vol. 91, pgs. 153-157 (1994)) and compared with the biological titer (pfu/ml) determined using 293 cell monolayers as described in Mittereder, et al., J. Virol., Vol. 70, pgs. 7498-7509 (1996). The ratio of total particles to infectious particles (particles/pfu) was calculated. DNA was isolated from each vector and digested with DraI, ScaI, or EcoRI and BamHI to confirm the identity of each. The schematic diagrams of Av9LacZ4 and parental, Av1LacZ4 vectors are shown schematically in FIG. 1.

Expression of fiber constructs in baculovirus. As described previously (Stevenson, 1995), the baculovirus expression system (Clontech, Palo Alto, Calif.) was used to generate fiber proteins for receptor binding studies. Recombinant baculoviral vectors were used which expressed either the Ad5 fiber or Ad3 fiber proteins. Spodoptera frugiperda cells (Sf21) were cultured as monolayers at 27° C. in Grace's supplemented insect cell medium containing 10% HIFBS, 100 Units/ml penicillin, 100 μg/ml streptomycin sulfate, and 2.5 μg/ml of amphotericin B. Large scale infections of Sf21 cells with each recombinant fiber baculovirus were carried out and fiber containing cell lysates were prepared as described (Stevenson, 1995).

The Adenovirus 5 fiber protein was purified from the Sf21 cell lysates as described previously (Stevenson, 1995). Briefly, the Adenovirus 5 fiber trimer was purified to homogeneity using a two-step purification procedure utilizing a DEAE-sepharose column, and then a Superose 6 gel filtration column equilibrated in PBS using an FPLC system (Pharmacia). Protein concentrations of the purified Adenovirus 5 fiber trimer and the insect cell lysates containing the Adenovirus 3 fiber (3F/CL) were determined by the bicinchoninic acid protein assay (Pierce, Rockford, Ill.) with bovine serum albumin (BSA) as the assay standard.

The expression of fiber proteins was verified by sodium dodecyl sulfate (SDS)-4/15% polyacrylamide gel electrophoresis (PAGE) under denaturing conditions and Western immunoblot analysis. The proteins were transferred to a polyvinylidene difluoride (PVDF) membrane by use of a small transblot apparatus (Biorad, Hercules, Calif.) for 30 minutes at 100 volts. After the transfer was completed, the PVDF membrane was stained transiently with Ponceau red and the molecular weight standards were marked directly on the membrane. Molecular weight standards used ranged from 200 to 14 kDa (Biorad). Nonspecific protein binding sites on the PVDF membrane were blocked using a 5% dried milk solution in 10 mM Tris, pH7.4 containing 150 mM NaCl, 2 mM EDTA 0.04% Tween-20 for one hour at room temperature or overnight at 4° C. The membrane then was incubated for one hour at room temperature with a 1:10,000 dilution of the primary anti-Adenovitus 2 fiber monoclonal antibody, 4D2-5 (ascites kindly provided by Dr. J. Engler, University of Alabama) or with 70 μg/ml of a partially purified anti-Adenovirus 3 fiber specific rabbit polyclonal antibody generated against the baculoviral expressed Adenovirus 3 fiber head domain (Stevenson, 1995). The membrane was developed with either a 1:10,000 dilution of the secondary sheep anti-mouse IgG horseradish peroxidase (HRPO)-conjugated antibody (Amersham Lifesciences, Arlington, Ill.) or with a 1:2000 dilution of donkey anti-rabbit IgG-HRPO using an enhanced chemiluminescence system (Amersham Lifesciences). The membrane was exposed to film for approximately 3 to 60 seconds.

Production of an anti-Adenovirus 3 fiber specific antiserum. The fiber head region of the Adenovirus 3 fiber was expressed using the baculoviral expression system as described (Stevenson, 1995). The insect cell lysate containing the Adenovirus 3 fiber head was used for immunizations of New Zealand White rabbits according to standard protocols (Lofstrand Labs Ltd, Gaithersburg, Md.). The IgG fraction was isolated and was applied to an affinity column containing covalently bound insect cell lysate proteins. The unbound fraction from this affinity column was obtained and tested for immunoreactivity against the Adenovirus 5, Adenovirus 3, and chimeric, 5TS3H fiber proteins using Western blot analysis.

Competitive viral transduction assay. The receptor tropism of the recombinant adenoviruses was evaluated using a viral transduction assay in the presence of fiber protein competitors. Monolayers of HeLa cells (ATCC CCL 2) cultured in DMEM with 10% HIFBS, 100 Units/ml penicillin, and 100 μg/ml streptomycin sulfate contained in 12 well dishes were incubated with various dilutions of either purified Adenovirus 5 fiber trimer protein (0.05 μg/ml up to 100 μg/ml) or with an insect cell lysate containing the Adenovirus 3 fiber (100 μg/ml up to 2000 μg/ml) for one hour at 37° C. in a total volume of 0.2 ml of DMEM, 2% HIFBS. The chimeric fiber Av9LacZ4 or parental, Av1LacZ4 adenoviral vectors were then added in a total volume of 5 μl to achieve a total particle per cell ratio of 100 by dilution of the virus into DMEM, 2% HIFBS. Virus transductions were carried out for 1 hour at 37 degrees. C. The monolayers were washed once with PBS, 1 ml of DMEM, 109 HIFBS was added per well, and the cells were incubated for an additional 24 hours to allow for β-galactosidase expression. The cell monolayers then were fixed using 0.56 glutaraldehyde in PBS and then were incubated with 1 mg/ml 5-bromo-4-chloro-3-indolyl-b-D-galactoside (X-gal), 5 mM potassium ferrocyanide, 2 mM MgCl.sub.2 in 0.5 ml PBS. The cells were stained approximately 24 hours at 37° C. The monolayers were washed with PBS and the average number of blue cells per high power field were quantitated by light microscopy using a Zeiss ID03 microscope, three to five fields were counted per well. The average number of blue cells per high power field was expressed as a percentage of the control which did not contain competitor fiber protein. Each concentration of competitor was carried out in triplicate and the average percentage .+−. standard deviation was expressed as a function of added competitor fiber protein. Each experiment was carried out three to four times and data from a representative experiment is shown.

Cell Culture. The transduction efficiency of Av9LacZ4 and Av1LacZ4 was surveyed on a panel of human cell lines. HeLa, MRC-5 (ATCC CCL-171), FaDu (ATCC HTB 43), and THP-1 (ATCC TIB-202) cells were obtained from the ATCC and cultured in the recommended medium. Human umbilical vein endothelial cells (HUVEC, CC-2517) and coronary artery endothelial cells (HCAEC, CC-2585) were obtained from the Clonetics Corporation (San Diego, Calif.) and cultured in the recommended medium. Each cell line was transduced with the chimeric fiber Av9LacZ4 or the wild type, Av1LacZ4 adenoviral vectors at 0, 10, 100, and 1000 total particles per cell for one hour at 37° C. in a total volume of 0.2 ml of culture medium containing 2% HIFBS. The cell monolayers were then washed once with PBS and 1 ml of the appropriate culture medium containing 10% HIFBS was added. THP-1 cells were incubated with the indicated concentration of vector for one hour at 37 degrees. C. in a total volume of 0.2 ml of culture medium containing 2% HIFBS, and then 1 ml of complete medium containing 10% HIFBS was added. The cells were incubated for 24 hours to allow for β-galactosidase expression. The cell monolayers were then fixed and stained with X-gal as described above. The incubation of each cell line in the X-gal solution varied from 1.5 hours up to 24 hours depending on the amount background staining found in the mock infected wells. The percent transduction was determined by light microscopy by counting the number of transduced, blue cells per total cells in a high power field using a Zeiss ID03 microscope, three to five fields were counted per well. Each vector dose was carried out in triplicate and the average percent transduction per high power field (mean.+−.sd, n=3 wells) was determined and expressed as a function of added vector. Each cell line was transduced at least three times and the data represents the mean percent transduction .+−. standard deviation from three independent experiments.

Results

Construction of an adenovirus vector containing a chimeric Ad5/Ad3 fiber gene. It was shown previously using chimeric fiber proteins expressed in vitro and in insect cells that the receptor specificity of the adenovirus fiber protein can be altered by exchanging the head domain with another serotype which recognizes a different receptor (Stevenson, 1995). To generate an adenoviral vector particle with an altered receptor specificity, the chimeric fiber gene containing the Adenovirus 3 fiber head domain fused to the Adenovirus 5 fiber tail and shaft, 5TS3H, was incorporated within the adenoviral genome of Av1LacZ4. For the precise replacement of the wild type Adenovirus 5 fiber gene, a shuttle plasmid was constructed which contained the last 8886 bp of the Ad d1327 genome from 73.9 to 100 map units including the Adenovirus 5 fiber gene, E4 and the right ITR. This shuttle plasmid was used for incorporation of modified fiber genes into the backbone of an E1 and E3 deleted adenoviral vector Av1LacZ4 via homologous recombination. This strategy replaces the native Adenovirus 5 fiber with the chimeric 5TS3H fiber sequences cloned within the prep5TS3Ha shuttle plasmid. The resulting vector, Av9LacZ4 contains the nuclear targeted beta-galactosidase cDNA and the Adenovirus 3 fiber head domain. This approach will allow for any modification to the native fiber sequence to be incorporated within the adenoviral genome.

Both the parental, Av1LacZ4 and the chimeric fiber Av9LacZ4 vectors are shown schematically in FIG. 1. The Adenovirus 3 fiber head region introduces additional DraI and ScaI restriction enzyme sites within the Av1LacZ4 genome which were used to identify the recombinant virus. Plaques which yielded the predicted DraI and ScaI diagnostic fragments as indicated in FIG. 1A were selected and expanded. Genomic DNA isolated from the purified chimeric fiber, Av9LacZ4 and the parental, Av1LacZ4 viruses was analyzed by restriction enzyme digestion and agarose gel electrophoresis (FIG. 1B). The expected DNA fragments were obtained for both the Av9LacZ4 and wild type, Av1LacZ4 viruses. Diagnostic 18.4 and 3.2 kb fragments were found after ScaI digestion of the Av9LacZ4 genomic DNA (FIG. 1B, lane 4) indicating the presence of the Adenovirus 3 fiber head domain. DraI restriction endonuclease digestion of Av9LacZ4 also confirmed the presence of the Adenovirus 3 fiber head domain as indicated by the 8.0 and 2.8 kb diagnostic fragments (FIG. 1B, lane 5). EcoRI and BamHI digestion produced an identical restriction pattern for both vectors as expected (FIG. 1B, lanes 3 and 6). Southern blot analysis using the Adenovirus 3 fiber head probe demonstrated the expected hybridization pattern for all restriction endonuclease digestions for both vectors (FIG. 1C). The 18.4 and 3.2 kb ScaI and the 8.0 and 2.8 kb DraI diagnostic fragments of Av9LacZ4 hybridized with the Adenovirus 3 fiber head probe (FIG. 1C, lanes 4 and 5). The 6.6 kb EcoRI/BamHI fragment which contains the full length 5TS3H fiber gene was also detected (FIG. 1C, lane 6). Southern blot analysis using the Adenovirus 5 fiber head probe (data not shown) demonstrated the expected hybridization pattern for Av1LacZ4 and confirmed that the chimeric fiber Av9LacZ4 virus preparation was free of parental, Av1LacZ4 virus.

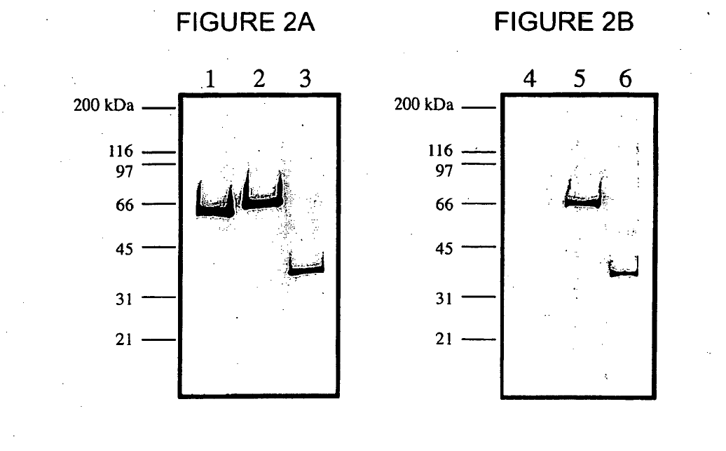

Characterization of adenoviral particles containing the Ad5/Ad3 chimeric fiber. Expression and assembly of the chimeric 5TS3H fiber protein into the adenoviral capsid was examined by Western Blot analysis of CsCl purified virus stocks. An equivalent number of the parental (Av1LacZ4) and chimeric (Av9LacZ4) particles were subjected to 4/1596 SDS PAGE under denaturing conditions. A control virus containing a full length Ad3 fiber was also analyzed. Western immunoblot analysis was carried out using an anti-fiber monoclonal antibody, 4D2-5 (FIG. 2A) and a rabbit polyclonal antibody specific for the Ad3 fiber head domain (FIG. 2B). The 4D2-5 antibody recognizes a conserved epitope located within the N-terminal tail domain of the fiber protein (Hong, et al., Embo. J., Vol. 14, pgs. 4714-4727 (1995)) and reacts with both the Adenovirus 5 (5F) and the Adenovirus 3 (3F) fiber proteins (Stevenson, 1995). As shown in FIG. 2A, the Av1LacZ4 (lane 1) and Av9LacZ4 (lane 2) viruses contain fiber proteins of approximately 62 to 63 kDa which react with the 4D2-5 antibody while the Adenovirus 3 fiber virus contains a fiber protein of approximately 35 kDa (FIG. 2A, lane 3). The presence of the Adenovirus 3 fiber head (3FH) domain within the 5TS3H chimeric fiber was confirmed by Western Blot analysis using a rabbit polyclonal antibody specific for the Adenovirus 3 fiber. The rabbit anti-3FH polyclonal antibody did not bind to the Adenovirus 5 fiber protein in Av1LacZ4 and was specific for the 35 kDa, Adenovirus 3 fiber protein in the control virus (FIG. 2B, lane 6) and the Adenovirus is fiber head domain contained within the chimeric 5TS3H fiber protein in Av9LacZ4 (FIG. 2B, lane 5).

The biological titers and particle numbers of the chimeric fiber (Av9LacZ4) and parental (Av1LacZ4) adenoviruses were compared. Biological titers determined using 293 cell monolayers indicated plaque forming titers of 2.6 and 4.5.times.10.sup.10 pfu/ml for the Av1LacZ4 and Av9LacZ4 viral preparations, respectively. The total particle concentrations were determined spectrophotometrically and were 1.45 and 1.25 times.10.sup.12 particles/ml for Av1LacZ4 and Av9LacZ4, respectively. Thus, the ratio of particle number to pfu titer was similar for both viruses, 55.8 versus 27.8 total particles/pfu, respectively. An increased ratio of particle number to infectious titer has previously been reported for Adenovirus 3 compared to Adenovirus 2 (Defer, et al., J. Virol., Vol. 64, pgs. 3661-3673 (1990)); however, the replacement of the Adenovirus 5 fiber head domain with the Adenovirus 3 fiber head domain did not adversely affect the cellular production of the adenovirus containing the chimeric fiber protein or significantly change the ratio of total physical to infectious particles.

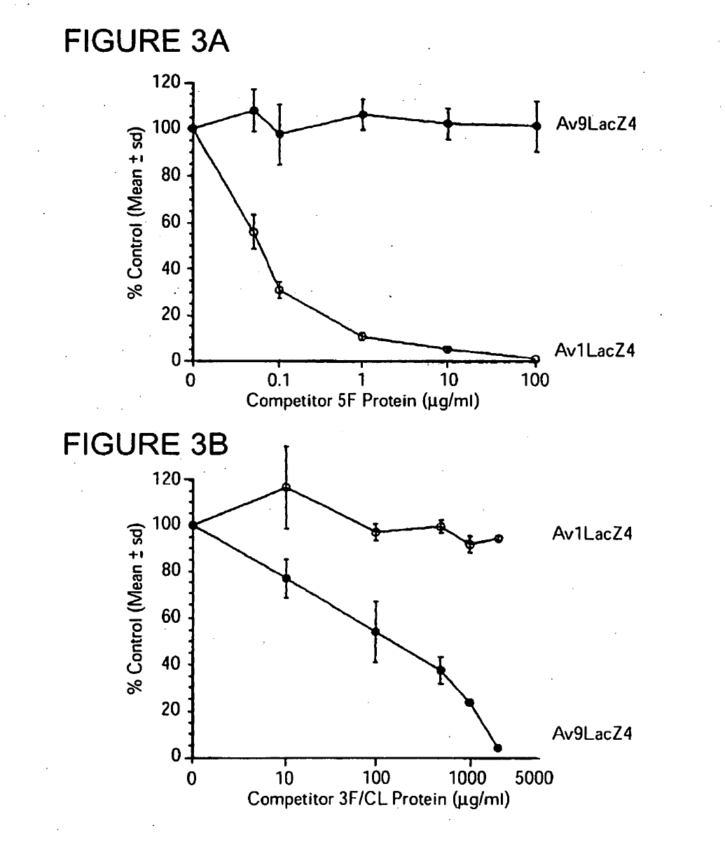

Receptor binding specificity of the modified Ad5/Ad3 fiber adenovirus. To evaluate the receptor binding properties of the chimeric fiber vector compared to the native Adenovirus 5 fiber vector, transduction experiments were carried out in the presence of recombinant fiber protein competitors. Cells were preincubated with purified Adenovirus 5 fiber protein or with an insect cell lysate containing the Adenovirus 3 fiber protein prior to transduction with the chimeric fiber or native Adenovirus 5 fiber vector. FIG. 3 shows the results of transduction experiment 3 in which HeLa cells were incubated with increasing amounts of Adenovirus 5 fiber protein (FIG. 3A) or with the Adenovirus 3 fiber competitor (FIG. 3B) prior to transduction with the Av9LacZ4 or Av1LacZ4 vectors. Transduction of HeLa cells with Av1LacZ4 decreased with increasing amounts of Adenovirus 5 fiber trimer protein, with maximal competition occurring between 0.1 to 1.0 mug/ml. In contrast, the purified Adenovirus 5 fiber trimer did not block the transduction of the Av9LacZ4 chimeric fiber adenovirus. These results confirm that the wild type, Av1LacZ4 and Av9LacZ4 chimeric fiber vectors bind to different cell surface receptors. This conclusion was supported by the reciprocal experiment shown in FIG. 3B. Increasing concentrations of the Adenovirus 3 fiber competitor decreased the AV9LacZ4 transduction of HeLa cells but did not influence transduction with the wild type, Av1LacZ4 vector. The competition between the Adenovirus 3 fiber competitor and Av9LacZ4 was specific since control experiments carried out with insect cell lysates which did not contain the Adenovirus 3 fiber protein did not result in competition (data not shown). These results indicate that transduction of HeLa cells by Av9LacZ4 is mediated by the chimeric fiber protein which interacts with the Adenovirus 3 receptor. Thus, the modification of the Adenovirus 5 fiber head domain has resulted in a change in receptor tropism of an adenoviral vector.

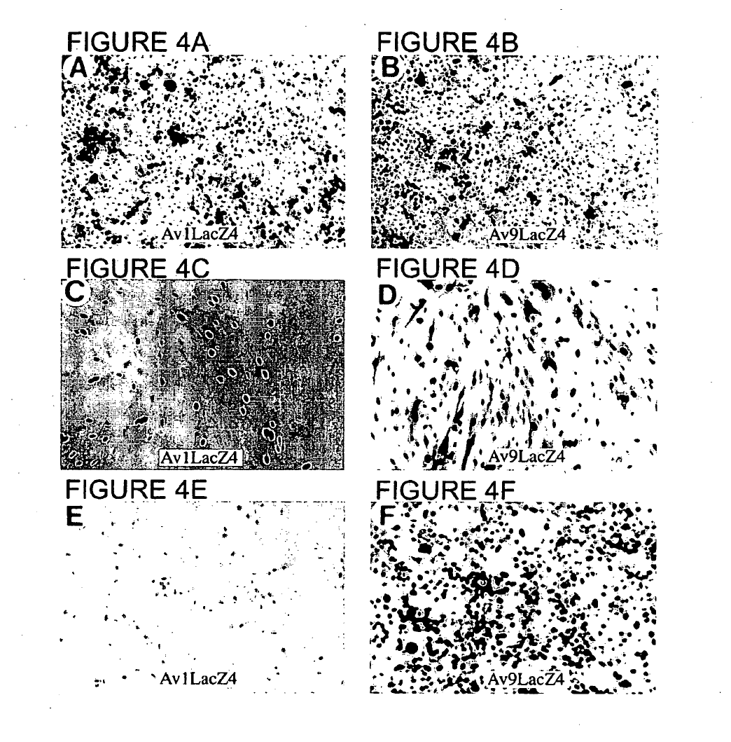

Transduction of human cell lines by the chimeric fiber vector. Because the identity of the Adenovirus 5 and Adenovirus 3 receptors is unknown, there is relatively little information available concerning their cellular distribution. It was hypothesized that differential expression of the Adenovirus 5 and Adenovirus 3 receptors on different human cells might be reflected in the differential transduction by the parental, Av1LacZ4 and chimeric fiber, Av9LacZ4 vectors. The transduction properties of a number of human cell lines by the two vectors was investigated. Several cell lines were included which had been identified as negative for Adenovirus 5 fiber adenovirus receptor binding (Haung, et al., J. Virol., Vol. 70, pgs. 4502-4508 (1996); Stevenson, 1995) and/or refractory to Av1LacZ4 infection (unpublished data). Cells ware infected with the chimeric fiber, Av9LacZ4 or the wild type, Av1LacZ4 adenovirus at particle per cell ratios of 0, 10, 100, and 1000 in a total volume of 0.2 ml of culture medium. 24 hours later the cells were stained with X-gal as hereinabove described. Shown in FIG. 4 are representative photographs of the Av1LacZ4 and Av9LacZ4 transduction of HeLa cells (FIGS. 4A and 4B), MRC-5, a human embryonic lung fibroblast cell line (FIGS. 4C and 4D), and FaDu, a human squamous cell carcinoma line (FIGS. 4E and 4F) monolayers at the 1000 virus particles per cell dose. Both vectors transduced HeLa cell monolayers with similar efficiencies. In contrast, differential transduction of the MRC-5 and FaDu cell lines was found. Both the MRC-5 and FaDu cells were relatively refractory to Av1LacZ4 transduction but were readily transduced with Av9LacZ4.

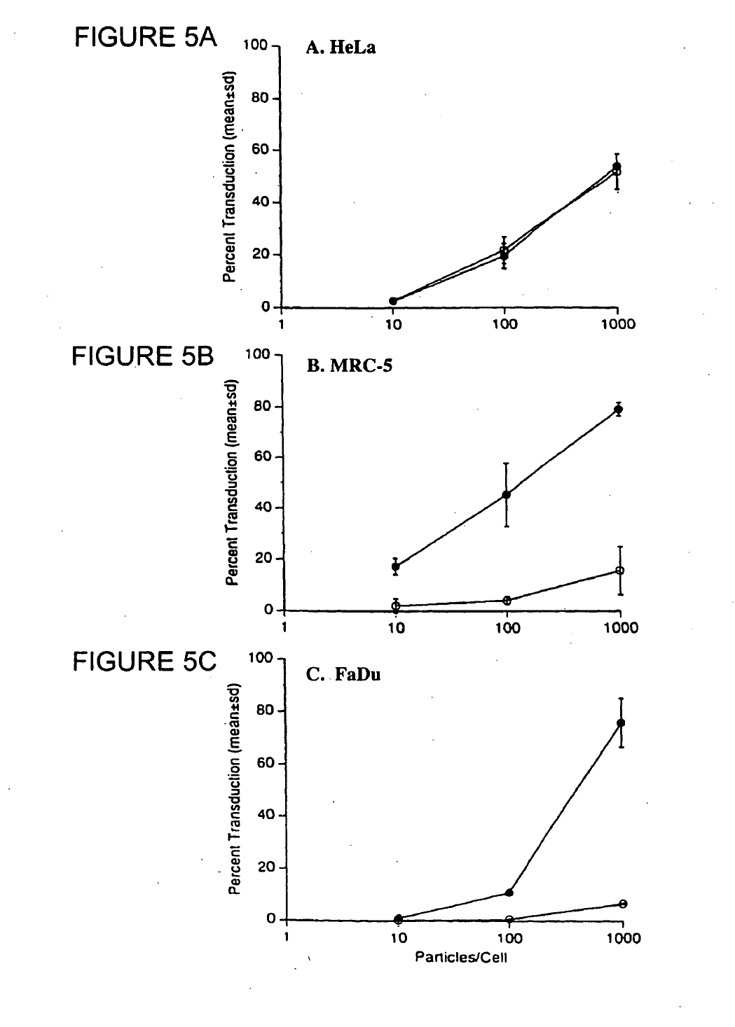

The percent transduction of each cell line was quantitated and the fraction of HeLa, MRC-5, and FaDu cells transduced as a function of dose is shown in FIG. 5. HeLa cells (FIG. 5A) were equally susceptible to transduction with both vectors indicating that both the Adenovirus 5 and Adenovirus 3 receptors are present on the cell surface. The MRC-5 (FIG. 5B) human embryonic lung cell line was efficiently transduced with the chimeric fiber, Av9LacZ4 vector. The percent transduction with Av9LacZ4 was dose dependent with approximately 80% transduction at the vector dose of 1000. Less efficient transduction of MRC-5 cells with Av1LacZ4 was observed suggesting that these cells either lack or express low levels of the Adenovirus 5 receptor. In contrast, the Adenovirus 3 receptor appears to be abundant on this cell type. The FaDu cell monolayers (FIG. 5C) were also transduced more efficiently with Av9LacZ4 with 75% of the cells transduced at the vector dose of 1000 compared to only 7% transduction achieved with Av1LacZ4 at the same vector dose.

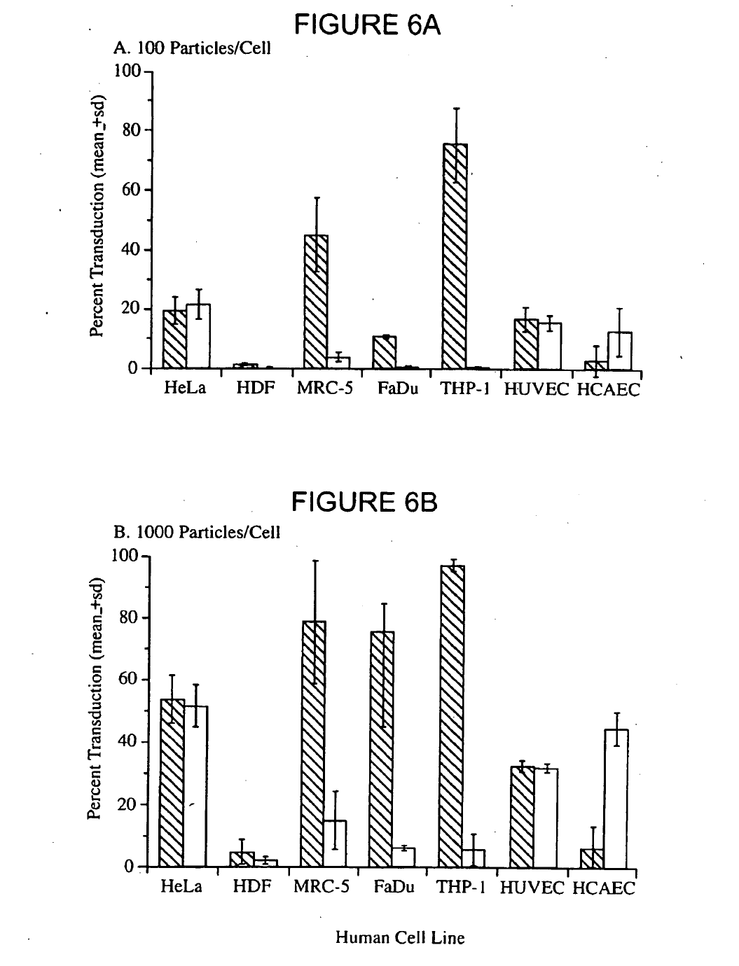

The transduction of a number of additional human cell lines were compared using Av1LacZ4 and Av9LacZ4. FIG. 6 summarizes data for each of the cell lines examined at the virus particle per cell ratios of 100 (FIG. 6A) and 1000 (FIG. 6B). The cell lines assessed in addition to the HeLa, MRC-5, and FaDu cell lines included HDF, human diploid fibroblasts; THP-1, human monocytes; HUVEC, human umbilical vein endothelial cells; and HCAEC, human coronary artery endothelial cells. Cells were infected with Av9LacZ4 or Av1LacZ4 adenoviral vectors at particle per cell ratios of 100 and 1000 and 24 hours later were stained with X-gal as hereinabove described. The fraction of transduced cells for each cell line at the indicated vector dose was determined. As shown previously, Hela cells were transduced at equivalent levels using both adenoviral vectors, while HDF cells were refractory to Av1LacZ4 as well as Av9LacZ4 transduction. HDF cells are negative for Adenovirus 5 fiber binding indicating that these cells lack or express low levels of the Adenovirus 5 receptor (Stevenson, 1995). The transduction data presented in FIG. 6 for HDF cells suggests that these cells lack or express low levels of the Adenovirus 3 receptor as well.

This analysis identified several human cell lines which were transduced differentially by the parental, Av1LacZ4 and the chimeric fiber, Av9LacZ4 vectors. MRC-5, FaDu, and THP-1 cells were efficiently infected with the recombinant vector containing the Adenovirus 3 fiber head in a dose dependent manner (FIGS. 6A and 6B), suggesting that the Adenovirus 3 receptor is more abundant than the Adenovirus 5 receptor on these cell types. At the vector dose of 1000 particles per cell approximately 450 of the HCAEC cells were transduced with the wild type fiber, Av1LacZ4 vector while only 7.3% were transduced with the chimeric fiber Av9LacZ4 vector. Venous endothelial cells (HUVEC) were equivalently transduced with both vectors. Differences in transduction of arterial and venous endothelial cells with Av1LacZ4 and Av9LacZ4 reveals the differential expression of the Adenovirus 3 and Adenovirus 5 receptors on cells derived from different regions of the vasculature. These data taken together demonstrate the differential expression of the Adenovirus 5 and Adenovirus 3 receptors on human cell lines derived from target tissues which are of potential clinical relevance.

Discussion

A major goal in gene therapy research is the development of vectors and delivery systems which can achieve efficient targeted in vivo gene transfer and expression. Vectors are needed which maximize the efficiency and selectivity of gene transfer to the appropriate cell type for expression of the therapeutic gene and which minimize gene transfer to other cells or sites in the body which could result in toxicity or unwanted side effects. Of the viral vectors under investigation for in vivo gene transfer applications, the adenovirus system has shown considerable promise and has undergone extensive evaluation in animal models as well as early clinical evaluation in lung disease and cancer. A key feature of adenovirus vectors is the efficiency of transduction and the resulting high levels of gene expression which can be achieved in vivo. This is derived from the ability to prepare high titer stocks of purified vector and from the remarkable efficiency of each of the steps in the adenoviral entry process leading to gene expression (Greber, et al., Cell, Vol. 75, pgs. 477-486 (1993)). Attachment of adenovirus particles to the cell is mediated by a high affinity interaction between the fiber protein and the cellular receptor (Philipson, et al. J. Virol., Vol. 2, pgs. 1064-1075 (1968)). Following binding, virion entry into many cell types is facilitated by an interaction between RGD peptide sequences in the penton base and the αvβ3 and αvβ5 integrins which act as co-receptors (Wickham, et al., Cell, Vol. 73, pgs. 303-319 (1993)). In the absence of the high affinity interaction of the fiber protein with its receptor, viral binding and transduction can still occur but with reduced efficiency. This fiber independent binding and transduction is believed to occur via a direct association between the penton base and cellular integrins (Haung, 1996). As the first step in the cellular transduction process, the interaction between the fiber protein and the cell is an attractive and logical target for controlling the cell specificity of transduction by adenoviral vectors. It has been shown that the receptor binding domain of the fiber protein resides within the trimeric globular head domain (Henry, et al., J. Virol., Vol. 68, pgs. 5239-5246 (1994); Louis, et al., J. Virol., Vol. 68, pgs. 4104-4106 (1994); Stevenson, 1995). The interaction of the fiber head domain with its receptor thus determines the binding specificity of adenoviruses. Consequently, manipulation of the fiber head domain represents an opportunity for control of the cell specificity of transduction by adenovirus vectors.

In order to test this concept experimentally, advantage was taken of the fact that adenoviruses of the group B and group C serotypes bind to different cellular receptors (Defer, 1990; Mathias, et al., J. Virol., Vol. 68, pgs. 6811-6814 (1994); Stevenson, 1995). Chimeric fiber proteins were prepared which exchanged the head domains of the Adenovirus 3 and Adenovirus 5 fiber proteins. Cell binding and competition studies with the recombinant chimeric fiber proteins confirmed the role of the fiber head domain in receptor binding and showed that an exchange of head domains resulted in a corresponding change of receptor specificity between the Adenovirus 3 and Adenovirus 5 receptors (Stevenson, 1995). In the present study, we have extended this analysis by the construction of an Adenovirus 5 based adenoviral vector, Av9LacZ4 which contains the fiber head domain from Adenovirus 3. The fiber modified vector was prepared by a gene replacement strategy using the .beta.-galactosidase expressing vector Av1LacZ4 as a starting point. A plasmid cassette containing the Adenovirus 5/Adenovirus 3 chimeric fiber gene, 5TS3H was used for homologous recombination with the Av1LacZ4 genome resulting in the precise substitution of the Adenovirus 5 fiber gene with the chimeric fiber gene containing the Adenovirus 3 fiber head to generate Av9LacZ4. Following plaque purification, molecular analysis of the recombinant vector genome provided confirmation of the fiber gene replacement in the vector. Western Blot analysis of purified vector particles using an antiserum specific for the Adenovirus 3 fiber verified the expression and assembly of the chimeric, 5TS3H fiber protein into functional adenoviral particles. The changed receptor specificity of the Av9LacZ4 chimeric fiber vector was confirmed by competition with recombinant fiber proteins which showed that transduction of 293 cells was effectively blocked by soluble Adenovirus 3 fiber but not by Adenovirus 5 fiber. This data confirms previous results obtained from binding experiments with recombinant fiber proteins and extends the analysis to intact adenovirus particles. Furthermore, the changed receptor specificity of the Av9LacZ4 vector establishes experimentally that the tropism of adenovirus vectors can be altered by manipulating the head domain.

The titer, yield, and ratio of physical to infectious particles of the fiber chimeric vector Av9LacZ4 and the parental Adenovirus 5, Av1LacZ4 vector were similar, thus indicating that the fiber head exchange did not alter significantly the growth properties of the vector on 293 cells. It has been reported that the infectivity of Adenovirus 3 is significantly less than that of Adenovirus 5, with Adenovirus 3 having a particle to PFU ratio approximately 20 times that of Adenovirus 5 (Defer, 1990). The similar infectivity of the Av9LacZ4 vector to the parental, Av1LacZ4 vector shows that the efficiency of entry of an Adenovirus 5 based vector via either the Adenovirus 5 or Adenovirus 3 receptor is similar. This suggests that the differences in the infectivity between Adenovirus 5 and Adenovirus 3 are not due to the use of a different receptor for binding and must reflect other differences between the two serotypes. The finding that the infectivity of the Av1LacZ4 and Av9LacZ4 vectors in 293 cells is similar leads to the important conclusion that the binding specificity of adenovirus vectors can be completely changed without affecting adversely the subsequent steps in entry and disassembly of the vector particles leading to nuclear gene delivery and expression. The implication of this result is that the function of the fiber receptor is primarily to promote efficient cellular attachment and that cell entry is an independent event which is not necessarily dependent on the molecule used for attachment. Therefore, it should be possible to modify the fiber protein to promote vector attachment to a range of different cell surface molecules without compromising the ability of the vector to enter the cell. This conclusion is supported by a recent report of a fiber modified adenovirus which binds to ubiquitously expressed cell surface proteoglycans and as a result has an extended cell tropism (Wickham, et al., Nature Biotechnology, Vol. 14, pgs. 1570-1573 (1996)). It should therefore be possible to construct other adenovirus vectors containing fiber proteins modified to contain ligands for cellular receptors which are expressed in a cell specific manner and as a result to achieve cell selective transduction.

The importance of the interaction between the fiber protein and the cellular fiber receptor for adenovirus infectivity is underscored by the fact that blockade of this interaction by soluble fiber protein results in the efficient inhibition of transduction (FIG. 3). Furthermore, cells which lack or express low levels of the cellular fiber receptor are inefficiently transduced and high levels of input vector are needed to achieve gene transfer (Haung, 1996). Recent clinical experience with adenoviral vectors in the treatment of cystic fibrosis lung disease has revealed a previously unsuspected resistance of human airway cells to transduction by Adenovirus 5 based vectors (Grub, et al., Nature, Vol. 371, pgs. 802-806 (1994); Zabner, et al. J. Virol., Vol. 70, pgs. 6994-7003 (1996)). It has been proposed that patterns of expression of both the .alpha.v integrins and the fiber attachment receptors may be involved in limiting transduction of human airway in vivo (Goldman, et al., J. Virol., Vol. 69, pgs. 5951-5958 (1995); Zabner, 1996). Evidence for a correlation between the level of .alpha.v integrin expression on human pulmonary epithelial cells and the efficiency of adenoviral vector transduction supports this hypothesis (Goldman, 1995).

The distribution of the Adenovirus 5 fiber attachment receptor on primary human cells is poorly characterized, largely due to the fact that its identity is unknown; however, it is increasingly clear that many human cell lines and a number of primary cells are refractory to transduction by Adenovirus 5 based vectors due to low levels or absence of the Adenovirus 5 fiber receptor. As noted previously, the Adenovirus 3 fiber receptor, while also as yet unknown, is clearly distinct from the Adenovirus 5 fiber receptor. Consequently, if differences in the pattern of expression of the two receptors exist, this should be reflected in a differential transduction efficiency by vectors which attach to either the Adenovirus 5 or Adenovirus 3 fiber receptors. In support of this hypothesis, several human cell lines have been identified, which were inefficiently transduced by the Adenovirus 5 vector, Av1LacZ4 and which could be transduced more efficiently by the chimeric fiber, Av9LacZ4 vector. These include a human head and neck tumor line FaDu, a human lung epithelial cell line MRC-5, and a human monocytic cell line THP-1. Transduction of HeLa cells and human umbilical vein endothelial cells (HUVEC) was equally efficient with both vectors. In contrast, human coronary artery endothelial cells (HCAEC) were more efficiently transduced by the Av1LacZ4 than by Av9LacZ4. Because the only difference between the two vectors is the identity of the fiber head domain, the differences observed in transduction are fiber dependent and must be a result of the differential expression of the two fiber receptors. The overlapping but distinct cellular distribution of the fiber receptors for Adenovirus 5 and Adenovirus 3 which is revealed by these results will likely be of practical value in designing vectors for transduction of specific human target cells. For example, the results obtained with the THP-1 cell line suggests that gene transfer to the monocyte/macrophage linage will be more efficient with vectors having the Adenovirus 3 receptor tropism than that of Adenovirus 5. Previous studies have demonstrated that human hematopoietic cells, monocytes, T-lymphocytes, and THP-1 cells were refractory to adenoviral vector transduction due to an apparent lack of Adenovirus 5 fiber receptors and were transduced only at high doses of input Adenovirus 5 vector (Haung, et al., J. Virol., Vol. 64, pgs. 2257-2263 (1995); Haung, 1996). The efficient transduction of monocytes with the Av9LacZ4 vector suggests that it may be useful in designing strategies for the treatment of cardiovascular disease and atherosclerosis by targeting macrophage cells in vessel wall lesions. Similarly, the FaDu cell data indicates that certain tumor cells will be transduced more effectively with the Av9LacZ4 vector than with Av1LacZ4.

The ability to modify adenoviral vectors to improve or enable transduction will increase the efficiency of adenoviral-mediated gene transfer. Modifications to the adenoviral fiber protein such as the head replacement strategy described in the present study is an approach which can lead to highly selective transduction of target cells. Head domains from other fiber proteins can be used to construct chimeric fibers which target vectors to alternative adenoviral receptors exploiting natural differences in the tropism of different adenoviral serotypes. Novel fiber proteins can also be constructed by replacement of the fiber head domain with other trimeric proteins, fusion of peptide sequences onto the Adenovirus 5 fiber C-terminus (Michael, et al., Gene Ther., Vol. 2, pgs. 660-668 (1995)) or addition of peptide ligands within exposed loop regions of the fiber head domain (Xia, et al., Structure, Vol. 2, pgs. 1259-1270 (1994)). These strategies will lead to the development of customized adenoviral vectors which selectively target specific cell types.

### Example 2

Transduction of Lung Carcinoma Cell Lines

The A549 lung carcinoma (ATCC No. CCL-185), H23 lung adenocarcinoma (ATCC No. CRL-5800), H358 lung bronchiolalveolar carcinoma (ATCC No. CRL-5807), H441 lung papillary adenocarcinoma (ATCC No. HTB-174), and H460 lung large cell carcinoma cell lines (ATCC No. HTB-177) were transduced with Av1LacZ4 or Av9LacZ4 at 100 or 1,000 particles per cell according to the procedure of Example 1. Transduction data are given in Table I below.

TABLE I

| | |
|---|---|
| Av9LacZ4 | Av1LacZ4 |
| particles/cell | particles/cell |

| Cell Line | 100 | 1,000 | 100 | 1,000 |
|---|---|---|---|---|
| A549 | ++ | ++++ | −/+ | +++ |
| H23 | +++ | +++ | +++ | +++ |
| H358 | +++ | ++++ | −/+ | ++ |
| H441 | ++ | ++++ | −/+ | −/+ |
| H460 | +++ | ++++ | ++ | +++ |

−/+ 0-25% transduction
++ 25-50% transduction
+++ 50-75% transduction
++++ 75-100% transduction

The above data suggests that an adenoviral vector having a head region from an Adenovirus 3 fiber can be employed for the transduction of lung carcinoma cells, and for the treatment of lung cancer.

### Example 3

### Transduction of Lymphoma and Leukemia Cells

U937 human histiocytic lymphoma cells (ATCC CRL-1593) were transduced with Av1LacZ4 or Av9LacZ4 at 100 or 1,000 particles/cell as described hereinabove in Example 1. Each experiment was carried out in triplicate, and the mean percentage of transduced cells was determined. No transduction was observed of U937 cells contacted with Av1LacZ4 at 100 particles/cell, and only 0.1% transduction of U937 cells was observed at 1,000 Av1LacZ4 particles/cell. In contrast, there was 3.4%.+−. 1.0% transduction of U937 cells with Av9LacZ4 at 100 particles/cells, and 9.2%.+−.0.4% transduction of U937 cells with Av9LacZ4 at 1,000 particles/cell.

In another experiment, K562 human chronic myelogenous leukemia cells (ATCC CCL243) were transduced with Av1LacZ4 or Av9LacZ4 at a multiplicity of infection (MOI) of 10, 50, or 100 according to the procedure of Example 1. Transduction results are given in Table II below.

TABLE II

| | |
|---|---|
| Av9LacZ4 | Av1LacZ4 |
| MOI | MOI |

| | | | |
|---|---|---|---|
| 10 | ++ | 10 | −/+ |
| 50 | ++++ | 50 | ++ |
| 100 | ++++ | 100 | +++ |

In another experiment, KG1 human bone marrow, acute myelogenous leukemia cells (ATCC CCL246) were transduced with Av1LacZ4 or Av9LacZ4 at a multiplicity of infection of 5, 10, 100, 500, or 1,000 according to the procedure of Example 1. Transduction data are given in Table III below.

TABLE III

| | |
|---|---|
| Av9LacZ4 | Av1LacZ4 |
| MOI | MOI |

| | | | |
|---|---|---|---|
| 5 | ++ | 5 | −/+ |
| 10 | +++ | 10 | −/+ |
| 100 | N/A | 100 | −/+ |
| 500 | +++ | 500 | N/A |
| 1,000 | N/A | 1000 | −/+ |

The results of the experiments in this example suggest that an adenoviral vector having a head region of the fiber of Adenovirus 3 may be employed in the treatment of leukemias or lymphomas.

### Example 4

Transduction of Human Smooth Muscle Cells. HISM human intestinal jejunum smooth muscle cells (ATCC CRL-1692) were transduced with Av1LacZ4 or Av9LacZ4 at 10, 100, or 1,000 particles/cell according to the procedure of Example 1. Each experiment was carried out in triplicate, and the percentages of transduced cells (mean+/−standard deviation) are given in Table IV below.

TABLE IV

| Particles/cell | Av9LacZ4 | Av1LacZ4 |
|---|---|---|
| 10 | 13.5 +/− 1.8 | 0.1 +/− 0.1 |
| 100 | 74.3 +/− 2.7 | 0.5 +/− 0.5 |
| 1,000 | 99.0 +/− 3.8 | 7.0 +/− 0.8 |

The above results suggest that an adenovirus having a head region of the fiber of Adenovirus 3 may be employed in the transduction of smooth muscle cells, such as smooth muscle cells of the digestive system or of the vasculature, and thus such adenoviruses may be useful in the treatment of a variety of disorders, such as the treatment of restenosis or of vascular lesions.

### Example 5

Transduction of Human Aortic Smooth Muscle Cells Human aortic smooth muscle cells (Clonetics) were transduced with Av1LacZ4 or Av9LacZ4 at 10, 100, or 1,000 particles/cell according to the procedure of Example 1. Each experiment was carried out in triplicate, and the percentages of transduced cells (mean+/−standard deviation) are given in Table V below.

TABLE V

| Particles/cell | Av9LacZ4 | Av1LacZ4 |
|---|---|---|
| 10 | 2.5 +/− 1.1 | 0 +/− 0 |
| 100 | 11.2 +/− 3.3 | 0.63 +/− 0 |
| 1,000 | 43.8 +/− 5.8 | 0.34 +/− 0.1 |

The above data suggest that an adenoviral vector having the head region of the fiber of Adenovirus 3 may be employed in the treatment of restenosis following angioplasty for the transduction of vascular smooth muscle cells for the delivery of a therapeutic transgene for the inhibition of smooth muscle cell proliferation.

### Example 6

### Construction of Adenovirus Vectors Containing a Chimeric Ad5/Ad3 and Ad5/Ad35 Fiber Genes the Express Human GM-CSF

Adenovirus vectors containing a chimeric Ad5/Ad35 or Ad5/Ad3 fiber gene that express human GM-CSF were generated in several steps. First, the full-length plasmid, pFLAd5, was constructed by combining the SmaI-linearized pAd5LtRtSmaI and the genomic DNA of Ad5 in E. coli. The resulting shuttle plasmid pFLAd5 comprises the Ad5 genome bordered by I-SceI sites. Next, pFLAd5 was digested with XhoI and the fragments containing the left and right terminal fragments of Ad5 were gel purified and self-ligated to generate pAd5-LtRtXhoI. The entire fiber-coding region was deleted using PCR and a recognition sequence for SwaI was inserted to generate pAd5-LtRtXhodelfiber. Combining XhoI-linearized pAd5LtRtXhodelfiber and the genomic DNA of CG0070 (U.S. application Ser. No. 10/925,205) generated the plasmid pFLAr20pAE2fhGmdelfiber containing the full-length CG0070 DNA minus fiber encoding region. A recombinant plasmid, pFBSE5T35H obtained from Genetic Therapy Inc., (GTI) and containing the gene encoding Ad5 fiber shaft and Ad35 fiber knob was digested with XbaI and EcoRV and the fragment containing the chimeric fiber encoding region was gel-purified using standard techniques. The plasmid, pFLAr20pAE2fGm-5T35H in which Ad5 shaft and Ad35 knob replacing Ad5 fiber-coding region was generated by combing SwaI linearized pFLAr20pAE2fhGmdelfiber with the gel-purified fragments in E. coli. A fiber chimeric oncolytic adenoviral vector, OV1191 was generated by digesting pFLAr20pAE2fGm-5T35H with I-SceI and transfecting into PER.C6 cells.

To generate the vector OV1192, a 3.6-kb EcoRI and KpnI restriction enzyme fragment containing the gene encoding chimeric fiber protein (Ad5 shaft and Ad3 knob) was obtained from genomic DNA of CRAd:hUPII-E1a-IRES-Eib/Fb5/3_{LL}-RGD and cloned into pBlueScript to generate pBlue-5T3H-RGD. Next, a 3.16-kb restriction enzyme fragment spanning the fiber-encoding region was obtained by digesting the pBlue-ST3H-RGD with EagI and KpnI. The gel-purified fragment was combined with SwaI-linearized pFLAr20pAE2fhGmdelfiber in E. coli to generate pFLAr20pAE2fhGM-5T3H-RGD. The resulting plasmid was digested with I-Scel and transfected into PER.C6 cells to generate OV1192.

Three additional chimeric fiber vectors that are similar to CG0070, OV1191 and 1192 in which an extra ATG located upstream of proper E1A ATG is deleted were generated in several steps. First, the left and right terminal KpnI restriction enzyme fragment obtained from pFLAr21pAe2fF were self-ligated to generate pAr21Lt&RtKpn-E2f. A 1.3-kb NheI-KpnI fragment was obtained from a full-length plasmid, pAr21pAE2fe (GTI). In this full-length plasmid an extra ATG upstream of E1A ATG has been deleted. This restriction enzyme fragment was used to replace the corresponding fragment from pAr21LtRt-Kpn-E2f to generate pAr21LtRtKpn-E2fe. Combining KpnI linearized pAr21LtRtKpn-E2fe with the genomic DNAs of CG0070, OV1191 and OV1192 generated three full-length plasmids, pFLAr20pAE2fe-5fiber, pFLAr20pAE2fe-5T35H, and pFLAr20pAE2fe-5T3H-RGD respectively. Linearization with I-Scel digestion and transfection of pFLAr20pAE2fe-5fiber, pFLAr20pAE2fe-5T35H, and pFLAr20pAE2fe-5T3H-RGD into PER.C6 cells generated OV1193, OV1194 and OV1195 respectively.

In addition, chimeric fiber adenoviral vectors in which E1A expression is placed under the control of hTERT promoter were generated. First, to replace the E2F-1 promoter with hTERT promoter, a NheI-KpnI restriction enzyme fragment of pAr21 LtRtKpn-E2f was replaced with a 1293-bp Nhe-KpnI fragment derived from pAr6pATrtexE3F. The resulting plasmid, pAr21LtRtKpn-Trtex, was linearized with KpnI and combined with genomic DNA derived from CGO070. OV1191, OV1192 to generate pFLAr20pATrtex-5fiber, pFLAr20pATrtex-5T35H and pFLAr20pATrtex-5T3H-RGD. Linearization with I-Scel enzyme digestion and transfection of pFLAr20pATrtex-5fiber, pFLAr20pATrtex-5T35H and pFLAr20pATrtex-5T3H-RGD into PER.C6 cells generated OV1196, OV1197 and OV1198 respectively.

Other adenoviruses used herein include CV802, which is a wild type Ad5 containing all wild type DNA sequence and used a positive control. Add1312 is a replication-defective vector with a deletion in the E1a gene and was used as a negative control vector.

All E1-containing vectors were purified using two rounds of cesium chloride density gradient centrifugation. Virus particle titers were determined by the spectrophotometric method as described previously (e.g., see Mittereder, et al 1996).

### Example 7

### Human Tumor Cell Lines and Cell Culture

Human head and neck cancer lines and human melanoma cell lines used for Ad5/Ad35 chimeric fiber vector studies are listed in Table VI.

TABLE VI
Tumor cell lines

| | | Source/catalog |
|---|---|---|
| Cell line | Description | number |
| Head and neck | | |
| cancer cell lines | | |
| A-253 | Human, epidermoid | ATCC, HTB-41 |
| | carcinoma | |
| A431 | Human, epidermoid | ATCC, CRL-2592 |
| | carcinoma | |
| FaDU | Human, squamous cell | ATCC, HTB-43 |
| | carcinoma (SQCC) | |
| SCC-9 | Human, tongue, SQCC | ATCC, CRL-1629 |
| SCC-15 | Human, tongue, SQCC | ATCC, CRL-1623 |
| Detroit 562 | Human, Pharyngeal | ATCC, CCL-138 |
| | carcinoma | |
| CAL 27 | Human, Tongue SQCC | ATCC, CRL-2095 |
| RPMI 2650 | Human, nasal septum, | ATCC, CCL-30 |
| | SQCC | |
| Melanoma Cell | | |
| lines | | |
| A375-luc | Human, skin, malignant | CRL-1619 |
| | melanoma | (modified to |
| | | express luciferase) |
| A2058 | Human, skin, malignant | ATCC, CRL-11147 |
| | melanoma | |
| C32 | Human, skin, malignant | ATCC, CRL-1585 |
| | melanoma | |
| SK-Mel-28 | Human, skin, malignant | ATCC, HTB-72 |
| | melanoma | |
| WM-266-4 | Human, skin, malignant | ATCC, CRL-1676 |
| | melanoma | |
| G-361 | Human, skin, malignant | ATCC, CRL-1424 |
| | melanoma | |

Human head and neck cancer lines and human melanoma cell lines listed in Table VI were cultured in RPMI 1640 medium containing 10% FBS.

### Example 8

### Density of Select Cell-Surface Receptors in and Transduction Efficiency of Human Tumor Cell Lines

In general, melanoma and head and neck cancer (HNC) cell lines are relatively less susceptible to Ad5 infection compared to fiber chimeric adenoviral vectors. To investigate the biological basis of the relative resistance of these cell lines to Ad5 but not to fiber chimeric vectors, cellular levels of receptors used by adenoviral vectors were determined. Cultured tumor cells were washed with PBS and detached from the plate with 0.025% trypsin, washed once with and resuspended in PBS (pH 7.4). The cells were incubated with mouse antibody directed against coxsackie-adenovirus receptor (CAR, Rmcb, Upstate biotechnology, Lake placid, NY), CD46 (Clone E4.3, BD Biosciences, Pharmingen, San Diego, Calif.)_{_{—}}ᵥ_{_{—}}₃ (Chemicon International, Temecula, Calif.) or _{_{—}}ᵥ_{_{—}}₅ (Chemicon International, Temecula, Calif.) for 30 min at 4° C. Subsequently, the cells were washed three times with PBS and incubated with FITC-conjugated secondary anti-mouse IgG (BD Biosciences, Pharmingen, San Diego, Calif.) for 30 min at 4° C. After washing with PBS, cells were suspended in PBS and analyzed by flow cytometry to determine percentage positive cells. The transduction efficiency mediated by fiber chimeric vectors expressing GFP was determined following infection of selected panel of melanoma and HNC cell lines. The cells were transduced at 50 viral particles per cell and incubated at 37° C. for 24 hours and percentage of transduction determined by flow cytometry. The data are shown in Tables VII and VIII.

TABLE VII
Selected cell-surface receptor expression and transduction efficiency
of human head and neck cancer cell lines by chimeric fiber adenoviruses
(% positive cells)

| | | | | | Detroit |
|---|---|---|---|---|---|
| Virus | A-253 | A431 | FaDu | SCC-9 | 562 |
| CAR | 16 | 22 | 2 | 6 | 3 |
| CD46 | 79 | 64 | 94 | 95 | 93 |
| Ad5GFP | 4 | 4 | 2 | 10 | 3 |
| Ad5GFP- | 21 | 32 | 59 | 56 | 35 |
| 5T35H | | | | | |
| Ad5GFP- | 22 | 34 | 5 | 6 | 33 |
| 5T3H-RGD | | | | | |

TABLE VIII
Selected cell-surface receptor expression in and transduction
efficiency of human melanoma cell lines by chimeric fiber
adenoviruses (% positive cells)

| | | A375- | SK-MEL- | | |
|---|---|---|---|---|---|
| Virus | WM-266-4 | luc | 28 | G361 | A2058 |
| CAR | 0.3 | 2 | 9 | 2 | 39 |
| CD46 | 14 | 69 | 5 | 4 | 25 |
| αvβ3 | 62 | 44 | 32 | 1 | 39 |
| αvβ5 | 2 | 28 | 2 | 1 | 3 |
| Ad5GFP | 2 | 18 | 10 | 4 | 17 |
| Ad5GFP- | 63 | 91 | 33 | 27 | 64 |
| 5T35H | | | | | |
| Ad5GFP- | 38 | 88 | 35 | 29 | 52 |
| 5T3H- | | | | | |
| RGD | | | | | |

These studies demonstrate that melanoma and HNC cell lines express low levels of CAR. In contrast, the levels of CD46 detected were relatively high, particularly for head and neck cancer cell lines. In addition, all five tested head and neck cancer cell lines had very low levels of _{_{—}}ᵥ_{_{—}}₃ and _{_{—}}ᵥ_{_{—}}₅ and therefore could not be detected by flow cytometry; however, the expression levels of _{_{—}}ᵥ_{_{—}}₃ integrins in a majority of melanoma cells were high. Thus, the relative susceptibility to fiber chimeric vectors and resistance to Ad5 is likely explained by high expression levels of CD46, the primary receptor for Ad3 and Ad35, and low level of CAR expression, the primary receptor for Ad5 on melanoma and HNC cells.

### Example 10

### In Vitro Ad5 and Chimeric Fiber Vector Mediated Transduction and Cytotoxicity of Human Cells

The in vitro cytotoxicity of Ad5 and Ad5 chimeric fiber vectors of the present invention was determined by exposing panel of tumor and normal cells to serial dilutions of virus for seven days. Cell viability was measured using an MTS cytotoxicity assay performed according to the manufacturer's instructions (CellTiter 96® AQᵤₑₒᵤₛ Non-Radioactive Cell Proliferation Assay, Promega, Madison, Wis.). Absorbance values are expressed as a percentage of uninfected control and plotted versus vector dose. A sigmoidal dose-response curve was fit to the data and EC₅₀ value calculated for each replicate using GraphPad Prism software, version 3.0. The EC₅₀ value is the dose of vector in particle per cell (PPC) that reduces the maximal light absorbance capacity of an exposed cell culture by 50%.

In vitro cytolytic potential of chimeric fiber oncolytic adenoviral vectors was tested in four representative head and neck cancer and melanoma cell lines. These data are summarized Tables 6 and 7.

TABLE 6
EC₅₀ values for representative head and neck cancer cell lines

| Virus | A-253 | SCC-9 | FaDu | A431 |
|---|---|---|---|---|
| CV802 | 16 | 12 | 72 | 31 |
| OV1193 | 205 | 59 | 323 | 291 |
| OV1194 | 20 | 6 | 23 | 55 |
| OV1195 | 7 | 4 | 34 | 20 |
| OV1191 | 20 | 39 | 23 | 49 |
| OV1192 | 14 | 24 | 60 | 30 |
| OV1196 | 189 | 40 | 206 | 302 |
| OV1197 | 13 | 5 | 9 | 28 |
| OV1198 | 11 | 1 | 25 | 38 |

The data presented in Table VI show that fiber chimeric vectors, OV1194 and OV1195 in which E2F(e) promoter is driving E1A were each approximately 10-fold more cytotoxic against four tested head and neck cancer cell lines compared to parental vector, OV1193 containing wild type Ad5 fiber. Similarly, the two other fiber chimeric vectors, OV1197 and OV1198 in which E1A expression was placed under the control of HTERT promoter were approximately 8- to 40-fold more cytotoxic against head and neck cancer cell lines compared corresponding parental vector, OV1196 carrying Ad5 wild type fiber. The EC₅₀ values for fiber chimeric vectors were approximately equivalent to wild type virus, CV802 suggesting that loss of potency in cytotoxicity by replacement of E1A promoter in fiber chimeric vectors is compensated by enhanced transduction. Based on these data and relatively high tumor selectivity of E2F(e) promoter, OV1194 and OV1195 were selected for further testing in few additional head and neck cancer cell lines.

In addition to head and neck cancers, melanomas also represent a potential target for fiber chimeric oncolytic vectors. The cytolytic potential of these oncolytic vectors was evaluated in a panel of melanoma cancer cell lines and the data are summarized in Table VIII.

TABLE VIII
EC₅₀ values for representative melanoma cell lines

| | | A375- | | |
|---|---|---|---|---|
| Virus | WM-266-4 | luc | G-361 | A2058 |
| CV802 | 667 | 45 | 52 | 16 |
| OV1193 | 882 | 377 | 177 | 2.9e+15 |
| OV1194 | 13 | 58 | 161 | |
| OV1195 | 9 | 19 | 40 | |
| OV1191 | 17 | 27 | 101 | |
| OV1192 | 25 | 14 | 83 | |
| OV1196 | 1253 | 102 | 88 | |
| OV1197 | 8 | 13 | 23 | |
| OV1198 | 9 | 7 | 18 | |

Similar to head and neck cancer cell lines, melanoma cell lines were more sensitive to fiber chimeric vectors, OV1194 and OV1195 compared to parental vector, OV1193. The data also showed that the EC₅₀ values of OV1194 and OV1195 were similar to wild type virus in three out of five tested cell lines and were approximately 100-fold more potent than wild type virus in two other tested cell lines. The cytotoxicy data presented in Tables VIII correlated well with the CAR, CD46 and integrin receptor density on these cell lines.

### Example 11

### Virus Production Assay

To assess the viral replication abilities, a few selected actively dividing tumor cell lines were infected with oncolytic vectors at 50 virus particles per cell (ppc). After 72 h, medium and cells were subjected to three freeze-thaw cycles and centrifuged to collect the supernatant. Serial log dilutions of supernatants were made and assayed for titer on 293 cells. For each cell line, the efficiency of oncolytic vector replication was expressed as TCID₅₀/ml.

TABLE IX
Virus production in representative head and neck cancer cell lines

| Cell Line | Onyx-015 | OV1193 | Ad-p53 | OV1194 | OV1195 |
|---|---|---|---|---|---|
| FaDu | 1.2E4 | 4.8E4 | 3.0E4 | 1.5E5 | 3.5E5 |
| SCC-9 | 1.1E5 | 1.3E5 | 2.2E4 | 9.2E5 | 7.2E5 |
| A253 | 6.5E4 | 9.3E4 | 5.4E4 | 2.2E5 | 2.2E5 |
| A431 | 1.2E5 | 1.1E5 | 2.5E4 | 2.2E5 | 2.1E5 |

TABLE X
Virus production in representative melanoma cell lines

| Cell Line | OV1193 | Ad-p53 | OV1194 | OV1195 |
|---|---|---|---|---|
| A375-luc | 1.3E5 | 4.1E5 | 1.6E6 | 1.6E6 |
| WM-266-4 | 7.4E4 | 7.4E3 | 6.5E5 | 1.1E5 |
| G-361 | 1.2E5 | 1.9E4 | 2.2E5 | 1.5E5 |
| SK-MEL-28 | 1.7E4 | 1.2E4 | 6.6E4 | 1.2E4 |

### Example 12

### Determination of Human GM-CSF Levels Expressed by Chimeric Fiber Ad Vectors

To evaluate human GM-CSF expression, cultured tumor cells were infected at 50 virus particles/cell, supernatants were collected 24 and 72 hours post infection and subjected to a commercially available ELISA assay (R&D Systems, Minneapolis, Minn.) to quantitate the total GM-CSF expressed. Cultured cell supernatants were diluted 10-fold to 1000-fold in assay buffer. Data were acquired on a spectrophotometer at 490 nm and the data were analyzed using the SoftMax software package. The standard curve for human GM-CSF typically had an R² value >0.995 and the sensitivity of the assay was typically 7.8 pg/mL. The amount of human GM-CSF expressed for representative chimeric fiber vectors for head and neck cancer cell lines is shown in Table XIA (24 hours) and Table XIB (72 hours) and for melanoma cell lines in Table XIIA (24 hours) and Table XIIB (72 hours).

TABLE XIA
Human GM-CSF expression in representative head and neck cancer
cell lines at 24 hours post-infection

| Virus | A-253 | A431 | Detroit 562 | FaDu | SCC-9 |
|---|---|---|---|---|---|
| OV1193 | 5 | 10 | 2 | 5 | 7 |
| OV1194 | 132 | 212 | 135 | 809 | 561 |
| OV1195 | 147 | 212 | 107 | 398 | 476 |

TABLE XIB
Human GM-CSF expression in representative head and neck cancer
cell lines at 72 hours post-infection

| Virus | A-253 | A431 | Detroit 562 | FaDu | SCC-9 |
|---|---|---|---|---|---|
| OV1193 | 285 | 81 | 84 | 131 | 154 |
| OV1194 | 659 | 384 | 360 | 1073 | 2011 |
| OV1195 | 468 | 376 | 323 | 1163 | 1469 |

TABLE XIIA
Human GM-CSF expression in representative melanoma cell lines at
24 hours post-infection

| Virus | A375 | WM-266-4 | A2058 | G-361 | SK-MEL-28 |
|---|---|---|---|---|---|
| CG0070 | 4 | 0.4 | 7 | 0.3 | 1.3 |
| OV1194 | 370 | 17 | 68 | 11 | 15 |
| OV1195 | 312 | 21 | 63 | 8 | 14 |

TABLE XIIB
Human GM-CSF expression in representative melanoma cell lines
at 72 hours post-infection

| Virus | A375 | WM-266-4 | A2058 | G-361 | SK-MEL-28 |
|---|---|---|---|---|---|
| CG0070 | 76 | 14 | 145 | 25 | 70 |
| OV1194 | 2074 | 188 | 672 | 130 | 146158 |
| OV1195 | 1347 | 246 | 302 | 177 | |

The results indicate the chimeric fiber adenoviral vectors of the present invention transduce human head and neck cancer cells and human melanoma cancer cells and can express high levels of human GM-CSF.

### Example 13

### In Vivo Efficacy of Ad5/Ad35 Chimeric Fiber Vectors in Xenograft Tumor Models

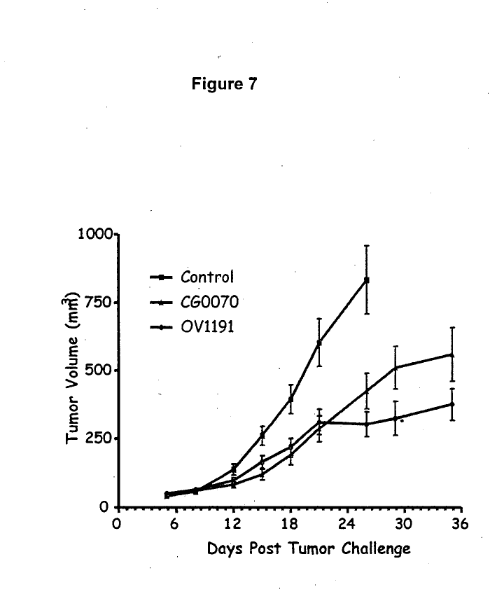

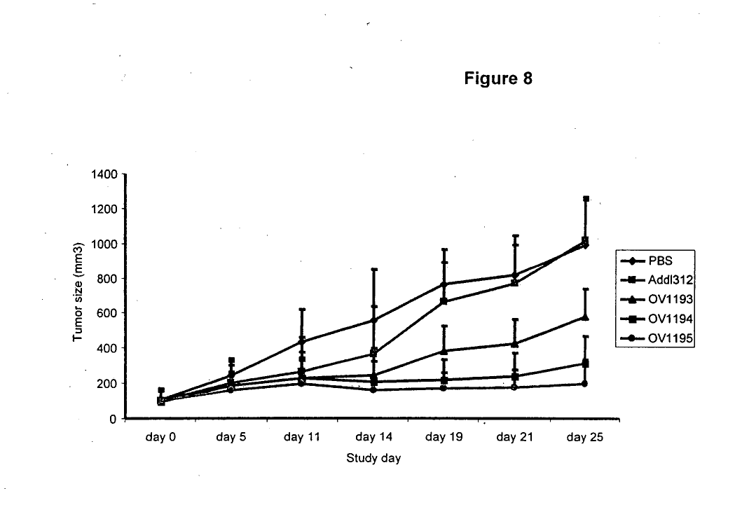

The efficacy of Ad5/Ad35 chimeric fiber vectors was evaluated in nude mice bearing FaDu (head and neck cancer) or A375-luc (melanoma) xenografts. Nude mice (Hsd:Athymic Nude-nu; Simonsen Labratories, Gilroy Calif.) were implanted with FaDu (5×10⁶ cells in 100-ul of HBSS) or A375-luc (2×10⁶ in 100-ul of HBSS) in the right flank. When tumors reached 50-150 mm³, mice were sorted into groups (n=10) and treated four times intra-tumorally with 1×10¹⁰ particles of viral agents or PBS in a 50-ul dose volume. The size of tumors were measured twice weekly in two dimensions, and the tumor volume was calculated as WX(L)²X_{—}/6. Mean tumor volume for each treatment group_SE mean was plotted versus days after vector injection. The results are depicted graphically in FIGS. 7 and 8.

Although the foregoing invention has been described in some detail by way of illustration and example for purposes of clarity and understanding, it will be apparent to those skilled in the art that certain changes and modifications may be practiced. Various aspects of the invention have been achieved by a series of experiments, some of which are described by way of the following non-limiting examples. Therefore, the description and examples should not be construed as limiting the scope of the invention, which is delineated by the appended claims. The disclosures of all patents, publications (including published patent applications), database accession numbers, and depository accession numbers referenced in this specification are specifically incorporated herein by reference in their entirety to the same extent as if each such individual patent, publication, database accession number, and depository accession number were specifically and individually indicated to be incorporated by reference.

It is to be understood, however, that the scope of the present invention is not to be limited to the specific embodiments described above. The invention may be practiced other than as particularly described and still be within the scope of the accompanying embodiments.

TABLE XIII
Brief Table Of The Sequences.

SEQ ID NO:1 E2F promoter (CGI) (270 bps)
TGGTACCATCCGGACAAAGCCTGCGCGCGCCCCGCCCCGCCATTGGCCGT
ACCGCCCCGCGCCGCCGCCCCATCCCGCCCCTCGCCGCCGGGTCCGGCGC
GTTAAAGCCAATAGGAACCGCCGCCGTTGTTCCCGTCACGGCCGGGGCAG
CCAATTGTGGCGGCGCTCGGCGGCTCGTGGCTCTTTCGCGGCAAAAAGGA
TTTGGCGCGTAAAAGTGGCCGGGACTTTGCAGGCAGCGGCGGCCGGGGGC
GGAGCGGGATCGAGCCCTCG
SEQ ID NO:2 hTERT promoter (CGI) (239 bps)
CGTGGCGGAGGGACTGGGGACCCGGGCACCCGTCCTGCCCCTTCACCTTC
CAGCTCCGCCTCCTCCGCGCGGACCCCGCCCCGTCCCGACCCCTCCCGGG
TCCCCGGCCCAGCCCCCTCCGGGCCCTCCCAGCCCCTCCCCTTCCTTTCC
GCGGCCCCGCCCTCTCCTCGCGGCGCGAGTTTCAGGCAGCGCTGCGTCCT
GCTGCGCACGTGGGAAGCCCTGGCCCCGGCCACCCCCGC
SEQ ID NO:3 hTERT promoter (GTI) (245 bps)
CCCCACGTGGCGGAGGGACTGGGGACCCGGGCACCCGTCCTGCCCCTTCA
CCTTCCAGCTCCGCCTCCTCCGCGCGGACCCCGCCCCGTCCCGACCCCTC
CCGGGTCCCCGGCCCAGCCCCCTCCGGGCCCTCCCAGCCCCTCCCCTTCC
TTTCCGCGGCCCCGCCCTCTCCTCGCGGCGCGAGTTTCAGGCAGCGCTGC
GTCCTGCTGCGCACGTGGGAAGCCCTGGCCCCGGCCACCCCCGCG
SEQ ID NO:4 CG5757 left end (2751 bps)
CATCATCAATAAATATACCTTATTTTGGATTGAAGCCAATATGATAATGA
GGGGGTGGAGTTTGTGACGTGGCGCGGGGCGTGGGAACGGGGCGGGTGAC
GTAGTAGTGTGGCGGAAGTGTGATGTTGCAAGTGTGGCGGAACACATGTA
AGCGACGGATGTGGCAAAAGTGACGTTTTTGGTGTGCGCCGGTGTACACA
GGAAGTGACAATTTTCGCGCGGTTTTAGGCGGATGTTGTAGTAAATTTGG
GCGTAACCGAGTAAGATTTGGCCATTTTCGCGGGAAAACTGAATAAGAGG
AAGTGAAATCTGAATAATTTTGTGTTACTCATAGCGCGTAATATTTGTCT
AGGGCCGCGGGGACTTTGACCGTTTACGTGACCGGTGGTACCATCCGGAC
AAAGCCTGCGCGCGCCCCGCCCCGCCATTGGCCGTACCGCCCCGCGCCGC
CGCCCCATCCCGCCCCTCGCCGCCGGGTCCGGCGCGTTAAAGCCAATAGG
AACCGCCGCCGTTGTTCCCGTCACGGCCGGGGCAGCCAATTGTGGCGGCG
CTCGGCGGCTCGTGGCTCTTTCGCGGCAAAAAGGATTTGGCGCGTAAAAG
TGGCCGGGACTTTGCAGGCAGCGGCGGCCGGGGGCGGAGCGGGATCGAGC
CCTCGACCGGTGACTGAAAATGAGACATATTATCTGCCACGGAGGTGTTA
TTACCGAAGAAATGGCCGCCAGTCTTTTGGACCAGCTGATCGAAGAGGTA
CTGGCTGATAATCTTCCACCTCCTAGCCATTTTGAACCACCTACCCTTCA
CGAACTGTATGATTTAGACGTGACGGCCCCCGAAGATCCCAACGAGGAGG
CGGTTTCGCAGATTTTTCCCGACTCTGTAATGTTGGCGGTGCAGGAAGGG
ATTGACTTACTCACTTTTCCGCCGGCGCCCGGTTCTCCGGAGGCGCCTCA
CCTTTCCCGGCAGCCCGAGCAGCCGGAGCAGAGAGCCTTGGGTCCGGTTT
CTATGCCAAACCTTGTACCGGAGGTGATCGATCTTACCTGCCACGAGGCT
GGCTTTCCACCCAGTGACGACGAGGATGAAGAGGGTGAGGAGTTTGTGTT
AGATTATGTGGAGCACCCCGGGCACGGTTGCAGGTCTTGTCATTATCACC
GGAGGAATACGGGGGACCCAGATATTATGTGTTCGCTTTGCTATATGAGG
ACCTGTGGCATGTTTGTCTACAGTAAGTGAAAATTATGGGCAGTGGGTGA
TAGAGTGGTGGGTTTGGTGTGGTAATTTTTTTTTAATTTTTACAGTTTTG
TGGTTTAAAGAATTTTGTATTGTGATTTTTTTAAAAGGTCCTGTGTCTGA
ACCTGAGCCTGAGCCCGAGCCAGAACCGGAGCCTGCAAGACCTACCCGCC
GTCCTAAAATGGCGCCTGCTATCCTGAGACGCCCGACGTCACCTGTGTCT
AGAGAATGCAATAGTAGTACGGATAGCTGTGACTCCGGTCCTTCTAACAC
ACCTCCTGAGATACACCCGGTGGTCCCGCTGTGCCCCATTAAACCAGTTG
CCGTGAGAGTTGGTGGGCGTCGCCAGGCTGTGGAATGTATCGAGGACTTG
CTTAACGAGCCTGGGCAACCTTTGGACTTGAGCTGTAAACGCCCCAGGCC
ATAAGGTGTAAACCTGTGATTGCGTGTGTGGTTAACGCCTTTGTTTGCTG
AATGGTCGACCGGTACCGTGGCGGAGGGACTGGGGACCCGGGCACCCGTC
CTGCCCCTTCACCTTCCAGCTCCGCCTCCTCCGCGCGGACCCCGCCCCGT
CCCGACCCCTCCCGGGTCCCCGGCCCAGCCCCCTCCGGGCCCTCCCAGCC
CCTCCCCTTCCTTTCCGCGGCCCCGCCCTCTCCTCGCGGCGCGAGTTTCA
GGCAGCGCTGCGTCCTGCTGCGCACGTGGGAAGCCCTGGCCCCGGCCACC
CCCGCACCGGTCGACGCGCTGCGGCTGCTGTTGCTTTTTTGAGTTTTATA
AAGGATAAATGGAGCGAAGAAACCCATCTGAGCGGGGGGTACCTGCTGGA
TTTTCTGGCCATGCATCTGTGGAGAGCGGTTGTGAGACACAAGAATCGCC
TGCTACTGTTGTCTTCCGTCCGCCCGGCGATAATACCGACGGAGGAGCAG
CAGCAGCAGCAGGAGGAAGCCAGGCGGCGGCGGCAGGAGCAGAGCCCATG
GAACCCGAGAGCCGGCCTGGACCCTCGGGAATGAATGTTGTACAGGTGGC
TGAACTGTATCCAGAACTGAGACGCATTTTGACAATTACAGAGGATGGGC
AGGGGCTAAAGGGGGTAAAGAGGGAGCGGGGGGCTTGTGAGGCTACAGAG
GAGGCTAGGAATCTAGCTTTTAGCTTAATGACCAGACACCGTCCTGAGTG
TATTACTTTTCAACAGATCAAGGATAATTGCGCTAATGAGCTTGATCTGC
TGGCGCAGAAGTATTCCATAGAGCAGCTGACCACTTACTGGCTGCAGCCA
GGGGATGATTTTGAGGAGGCTATTAGGGTATATGCAAAGGTGGCACTTAG
GCCAGATTGCAAGTACAAGATCAGCAAACTTGTAAATATCAGGAATTGTT
GCTACATTTCTGGGAACGGGGCCGAGGTGGAGATAGATACGGAGGATAGG
GTGGCCTTTAGATGTAGCATGATAAATATGTGGCCGGGGGTGCTTGGCAT
GGACGGGGTGGTTATTATGAATGTAAGGTTTACTGGCCCCAATTTTAGCG
G
SEQ ID NO:5 (scs4)
CATCTGCAGCATGAAGCGCGCAAGACCGTCTGAAGATA
SEQ ID NO:6 (scs80)
CGTTGAAACATAACACAAACGAITCTTTATTCATCTTCTCTAATATAGGA
AAAGGTAAk
SEQ ID NO:7 (scs79)
TTACCTTTTCCTATATTAGAGAAGATGAATAAAGAATCGTTTGTGTTATG
TTTCAACG
SEQ ID NO:8 (scs81)
AGACAAGCTTGCATGCCTGCAGGACGGAGC
SEQ ID NO:9:
KKTK
SEQ ID NO:10
KLGTGLSFD
SEQ ID NO:11
GNLTSQNVTTVSPPLKKTK
SEQ ID NO:12
GTLQENIRATAPITKNN
SEQ ID NO:13 Nucleotide sequence of an ORF
encoding Ad35 fiber protein
ATGACCAAGAGAGTCCGGCTCAGTGACTCCTTCAACCCTGTCTACCCCTA
TGAAGATGAAAGCACCTCCCAACACCCCTTTATAAACCCAGGGTTTATTT
CCCCAAATGGCTTCACACAAAGCCCAGACGGAGTTCTTACTTTAAAATGT
TTAACCCCACTAACAACCACAGGCGGATCTCTACAGCTAAAAGTGGGAGG
GGGACTTACAGTGGATGACACTGATGGTACCTTACAAGAAAACATACGTG
CTACAGCACCCATTACTAAAAATAATCACTCTGTAGAACTATCCATTGGA
AATGGATTAGAAACTCAAAACAATAAACTATGTGCCAAATTGGGAAATGG
GTTAAAATTTAACAACGGTGACATTTGTATAAAGGATAGTATTAACACCT
TATGGACTGGAATAAACCCTCCACCTAACTGTCAAATTGTGGAAAACACT
AATACAAATGATGGCAAACTTACTTTAGTATTAGTAAAAAATGGAGGGCT
TGTTAATGGCTACGTGTCTCTAGTTGGTGTATCAGACACTGTGAACCAAA
TGTTCACACAAAAGACAGCAAACATCCAATTAAGATTATATTTTGACTCT
TCTGGAAATCTATTAACTGAGGAATCAGACTTAAAAATTCCACTTAAAAA
TAAATCTTCTACAGCGACCAGTGAAACTGTAGCCAGCAGCAAAGCCTTTA
TGCCAAGTACTACAGCTTATCCCTTCAACACCACTACTAGGGATAGTGAA
AACTACATTCATGGAATATGTTACTACATGACTAGTTATGATAGAAGTCT
ATTTCCCTTGAACATTTCTATAATGCTAAACAGCCGTATGATTTCTTCCA
ATGTTGCCTATGCCATACAATTTGAATGGAATCTAAATGCAAGTGAATCT
CCAGAAAGCAACATAGCTACGCTGACCACATCCCCCTTTTTCTTTTCTTA
CATTACAGAAGACGACGAATAA
SEQ ID NO:14 Amino acid sequence of Ad35 fiber:
323 amino acids in length, tail and knob regions
of the protein are underlined.
MTKRVRLSDSFNPVYPYEDESTSQHPFINPGFISPNGFTQSPDGVLTLKC
LTPLTTTGGSLQLKVGGGLTVDDTDGTLQENIRATAPITKNNHSVELSIG
NGLETQNNKLCAKLGNGLKFNNGDICIKDSINTLWTGINPPPNCQIVENT
NTNDGKLTLVLVKNGGLVNGYVSLVGVSDTVNQMFTQKTANIQLRLYFDS
SGNLLTEESDLKIPLKNKSSTATSETVASSKAFMPSTTAYPFNTTTRDSE
NYIHGICYYMTSYDRSLFPLNISIMLNSRMISSNVAYAIQFEWNLNASES
PESNIATLTTSPFFFSYITEDDE
SEQ ID NO:15 - a nucleic acid sequence encoding
an Ad5 fiber protein
ATGAAGCGCGCAAGACCGTCTGAAGATACCTTCAACCCCGTGTATCCATA
TGACACGGAAACCGGTCCTCCAACTGTGCCTTTTCTTACTCCTCCCTTTG
TATCCCCCAATGGGTTTCAAGAGAGTCCCCCTGGGGTACTCTCTTTGCGC
CTATCCGAACCTCTAGTTACCTCCAATGGCATGCTTGCGCTCAAAATGGG
CAACGGCCTCTCTCTGGACGAGGCCGGCAACCTTACCTCCCAAAATGTAA
CCACTGTGAGCCCACCTCTCAAAAAAACCAAGTCAAACATAAACCTGGAA
ATATCTGCACCCCTCACAGTTACCTCAGAAGCCCTAACTGTGGCTGCCGC
CGCACCTCTAATGGTCGCGGGCAACACACTCACCATGCAATCACAGGCCC
CGCTAACCGTGCACGACTCCAAACTTAGCATTGCCACCCAAGGACCCCTC
ACAGTGTCAGAAGGAAAGCTAGCCCTGCAAACATCAGGCCCCCTCACCAC
CACCGATAGCAGTACCCTTACTATCACTGCCTCACCCCCTCTAACTACTG
CCACTGGTAGCTTGGGCATTGACTTGAAAGAGCCCATTTATACACAAAAT
GGAAAACTAGGACTAAAGTACGGGGCTCCTTTGCATGTAACAGACGACCT
AAACACTTTGACCGTAGCAACTGGTCCAGGTGTGACTATTAATAATACTT
CCTTGCAAACTAAAGTTACTGGAGCCTTGGGTTTTGATTCACAAGGCAAT
ATGCAACTTAATGTAGCAGGAGGACTAAGGATTGATTCTCAAAACAGACG
CCTTATACTTGATGTTAGTTATCCGTTTGATGCTCAAAACCAACTAAATC
TAAGACTAGGACAGGGCCCTCTTTTTATAAACTCAGCCCACAACTTGGAT
ATTAACTACAACAAAGGCCTTTACTTGTTTACAGCTTCAAACAATTCCAA
AAAGCTTGAGGTTAACCTAAGCACTGCCAAGGGGTTGATGTTTGACGCTA
CAGCCATAGCCATTAATGCAGGAGATGGGCTTGAATTTGGTTCACCTAAT
GCACCAAACACAAATCCCCTCAAAACAAAAATTGGCCATGGCCTAGAATT
TGATTCAAACAAGGCTATGGTTCCTAAACTAGGAACTGGCCTTAGTTTTG
ACAGCACAGGTGCCATTACAGTAGGAAACAAAAATAATGATAAGCTAACT
TTGTGGACCACACCAGCTCCATCTCCTAACTGTAGACTAAATGCAGAGAA
AGATGCTAAACTCACTTTGGTCTTAACAAAATGTGGCAGTCAAATACTTG
CTACAGTTTCAGTTTTGGCTGTTAAAGGCAGTTTGGCTCCAATATCTGGA
ACAGTTCAAAGTGCTCATCTTATTATAAGATTTGACGAAAATGGAGTGCT
ACTAAACAATTCCTTCCTGGACCCAGAATATTGGAACTTTAGAAATGGAG
ATCTTACTGAAGGCACAGCCTATACAAACGCTGTTGGATTTATGCCTAAC
CTATCAGCTTATCCAAAATCTCACGGTAAAACTGCCAAAAGTAACATTGT
CAGTCAAGTTTACTTAAACGGAGACAAAACTAAACCTGTAACACTAACCA
TTACACTAAACGGTACACAGGAACAGGAGACACAACTCCAAGTGCATACT
CTATGTCATTTTCATGGGACTGGTCTGGCCACAACTACATTAATGAAATA
TTTGCCACATCCTCTTACACTTTTTCATACATTGCCCAAGAATAA
SEQ ID NO:16 Amino acid sequence of Ad 5 fiber
MKRARPSEDTFNPVYPYDTETGPPTVPFLTPPFVSPNGFQESPPGVLSLR
LSEPLVTSNGMLALKMGNGLSLDEAGNLTSQNVTTVSPPLKKTKSNINLE
ISAPLTVTSEALTVAAAAPLMVAGNTLTMQSQAPLTVHDSKLSIATQGPL
TVSEGKLALQTSGPLTTTDSSTLTITASPPLTTATGSLGIDLKEPIYTQN
GKLGLKYGAPLHVTDDLNTLTVATGPGVTINNTSLQTKVTGALGFDSQGN
MQLNVAGGLRIDSQNRRLILDVSYPFDAQNQLNLRLGQGPLFINSAHNLD
INYNKGLYLFTASNNSKKLEVNLSTAKGLMFDATAIAINAGDGLEFGSPN
APNTNPLKTKIGHGLEFDSNKAMVPKLGTGLSFDSTGAITVGNKNNDKLT
LWTTPAPSPNCRLNAEKDAKLTLVLTKCGSQILATVSVLAVKGSLAPISG
TVQSAHLIIRFDENGVLLNNSFLDPEYWNFRNGDLTEGTAYTNAVGFMPN
LSAYPKSHGKTAKSNIVSQVYLNGDKTKPVTLTITLNGTQETGDTTPSAY
SMSFSWDWSGHNYINEIFATSSYTFSYIAQE
SEQ ID NO:17 Nucleotide sequence of the gene
(ORF) encoding 5T35H fiber protein
ATGAAGCGCGCAAGACCGTCTGAAGATACCTTCAACCCCGTGTATCCATA
TGACACGGAAACCGGTCCTCCAACTGTGCCTTTTCTTACTCCTCCCTTTG
TATCCCCCAATGGGTTTCAAGAGAGTCCCCCTGGGGTACTCTCTTTGCGC
CTATCCGAACCTCTAGTTACCTCCAATGGCATGCTTGCGCTCAAAATGGG
CAACGGCCTCTCTCTGGACGAGGCCGGCAACCTTACCTCCCAAAATGTAA
CCACTGTGAGCCCACCTCTCAAAAAAACCAAGTCAAACATAAACCTGGAA
ATATCTGCACCCCTCACAGTTACCTCAGAAGCCCTAACTGTGGCTGCCGC
CGCACCTCTAATGGTCGCGGGCAACACACTCACCATGCAATCACAGGCCC
CGCTAACCGTGCACGACTCCAAACTTAGCATTGCCACCCAAGGACCCCTC
ACAGTGTCAGAAGGAAAGCTAGCCCTGCAAACATCAGGCCCCCTCACCAC
CACCGATAGCAGTACCCTTACTATCACTGCCTCACCCCCTCTAACTACTG
CCACTGGTAGCTTGGGCATTGACTTGAAAGAGCCCATTTATACACAAAAT
GGAAAACTAGGACTAAAGTACGGGGCTCCTTTGCATGTAACAGACGACCT
AAACACTTTGACCGTAGCAACTGGTCCAGGTGTGACTATTAATAATACTT
CCTTGCAAACTAAAGTTACTGGAGCCTTGGGTTTTGATTCACAAGGCAAT
ATGCAACTTAATGTAGCAGGAGGACTAAGGATTGATTCTCAAAACAGACG
CCTTATACTTGATGTTAGTTATCCGTTTGATGCTCAAAACCAACTAAATC
TAAGACTAGGACAGGGCCCTCTTTTTATAAACTCAGCCCACAACTTGGAT
ATTAACTACAACAAAGGCCTTTACTTGTTTACAGCTTCAAACAATTCCAA
AAAGCTTGAGGTTAACCTAAGCACTGCCAAGGGGTTGATGTTTGACGCTA
CAGCCATAGCCATTAATGCAGGAGATGGGCTTGAATTTGGTTCACCTAAT
GCACCAAACACAAATCCCCTCAAAACAAAAATTGGCCATGGCCTAGAATT
TGATTCAAACAAGGCTATGGTTCCTAAACTAGGAACTGGCCTTAGTTTTG
ACAGCACAGGTGCCATTACAGTAGGAAACAAAAATAATGATAAGCTAACT
TTGTGGACCGGAATAAACCCTCCACCTAACTGTCAAATTGTGGAAAACAC
TAATACAAATGATGGCAAACTTACTTTAGTATTAGTAAAAAATGGAGGGC
TTGTTAATGGCTACGTGTCTCTAGTTGGTGTATCAGACACTGTGAACCAA
ATGTTCACACAAAAGACAGCAAACATCCAATTAAGATTATATTTTGACTC
TTCTGGAAATCTATTAACTGAGGAATCAGACTTAAAAATTCCACTTAAAA
ATAAATCTTCTACAGCGACCAGTGAAACTGTAGCCAGCAGCAAAGCCTTT
ATGCCAAGTACTACAGCTTATCCCTTCAACACCACTACTAGGGATAGTGA
AAACTACATTCATGGAATATGTTACTACATGACTAGTTATGATAGAAGTC
TATTTCCCTTGAACATTTCTATAATGCTAAACAGCCGTATGATTTCTTCC
AATGTTGCCTATGCCATACAATTTGAATGGAATCTAAATGCAAGTGAATC
TCCAGAAAGCAACATAGCTACGCTGACCACATCCCCCTTTTTCTTTTCTT
ACATTACAGAAGACGACGAATAA
SEQ ID NO:18 Amino acid sequence of 5T35H fiber
(the tail and shaft derived from Ad5 and knob
region obtained from Ad35): 590 amino acids in
length, tail and knob regions of the protein are
underlined
MKRARPSEDTFNPVYPYDTETGPPTVPFLTPPFVSPNGFQESPPGVLSLR
LSEPLVTSNGMLALKMGNGLSLDEAGNLTSQNVTTVSPPLKKTKSNINLE
ISAPLTVTSEALTVAAAAPLMVAGNTLTMQSQAPLTVHDSKLSIATQGPL
TVSEGKLALQTSGPLTTTDSSTLTITASPPLTTATGSLGIDLKEPIYTQN
GKLGLKYGAPLHVTDDLNTLTVATGPGVTINNTSLQTKVTGALGFDSQGN
MQLNVAGGLRIDSQNRRLILDVSYPFDAQNQLNLRLGQGPLFINSAHNLD
INYNKGLYLFTASNNSKKLEVNLSTAKGLMFDATAIAINAGDGLEFGSPN
APNTNPLKTKIGHGLEFDSNKAMVPKLGTGLSFDSTGAITVGNKNNDKLT
LWTGINPPPNCQIVENTNTNDGKLTLVLVKNGGLVNGYVSLVGVSDTVNQ
MFTQKTANIQLRLYFDSSGNLLTEESDLKIPLKNKSSTATSETVASSKAF
MPSTTAYPFNTTTRDSENYIHGICYYMTSYDRSLFPLNISIMLNSRMISS
NVAYAIQFEWNLNASESPESNIATLTTSPFFFSYITEDDE
SEQ ID NO:19 Nucleotide sequence of an ORF
encoding Ad3 fiber protein nucleotides 205-1209
of GenBank Accession No. X01998.1
GGCCTTCGAGACCTCCTACCCATGAACTAATCATTGCCCCTACCTTACCC
AATCAAAATATTAATAAAGACACTTACTTGAAATCAGCAATACAGTCTTT
GTCAAAACTTTCTACCAGCAGCACCTCACCCTCTTCCCAACTCTGGTACT
CTAAACGTCGGAGGGTGGCATACTTTCTCCACACTTTGAAAGGGATGTCA
AATTTTATTTCCTCTTCTTTGCCCACAATCTTCATTTCTTTATCCCCAGA
TGGCCAAGCGAGCTCGGCTAAGCACTTCCTTCAACCCGGTGTACCCTTAT
GAAGATGAAAGCAGCTCACAACACCCATTTATAAATCCTGGTTTCATTTC
CCCTGACGGGTTCACACAAAGTCCAAACGGGGTTTTAAGTCTTAAATGTG
TTAATCCACTTACCACTGCAAGCGGCTCCCTCCAACTTAAAGTGGGAAGT
GGTCTTACAGTAGACACTACTGATGGATCCTTAGAAGAAAACATCAAAGT
TAACACCCCCCTAACAAAGTCAAACCATTCTATAAATTTACCAATAGGAA
ACGGTTTGCAAATAGAACAAAACAAACTTTGCAGTAAACTCGGAAATGGT
CTTACATTTGACTCTTCCAATTCTATTGCACTGAAAAATAACACTTTATG
GACAGGTCCAAAACCAGAAGCCAACTGCATAATTGAATACGGGAAACAAA
ACCCAGATAGCAAACTAACTTTAATCCTTGTAAAAAATGGAGGAATTGTT
AATGGATATGTAACGCTAATGGGAGCCTCAGACTACGTTAACACCTTATT
TAAAAACAAAAATGTCTCCATTAATGTAGAACTATACTTTGATGCCACTG
GTCATATATTACCAGACTCATCTTCTCTTAAAACAGATCTAGAACTAAAA
TACAAGCAAACCGCTGACTTTAGTGCAAGAGGTTTTATGCCAAGTACTAC
AGCGTATCCATTTGTCCTTCCTAATGCGGGAACACATAATGAAAATTATA
TTTTTGGTCAATGCTACTACAAAGCAAGCGATGGTGCCCTTTTTCCGTTG
GAAGTTACTGTTATGCTTAATAAACGCCTGCCAGATAGTCGCACATCCTA
TGTTATGACTTTTTTATGGTCCTTGAATGCTGGTCTAGCTCCAGAAACTA
CTCAGGCAACCCTCATAACCTCCCCATTTACCTTTTCCTATATTAGAGAA
GATGACTGACAACAAAAATAAAGTTCAACATTTTTTATTGAAATTCCTTT
TACAGTATTCGAGTAGTTATTTTGCCTCCCCCTTCCCATTTAACAGAATA
CACCAATCTCTCCCCACGCACAGCTTTAAA
SEQ ID NO:20 is the 319 amino acid sequence for
the Ad3 fiber protein from GenBank Accession No.
ERADF3.
MAKRARLSTSFNPVYPYEDESSSQHPFINPGFISPDGFTQSPNGVLSLKC
VNPLTTASGSLQLKVGSGLTVDTTDGSLEENIKVNTPLTKSNHSINLPIG
NGLQIEQNKLCSKLGNGLTFDSSNSIALKNNTLWTGPKPEANCIIEYGKQ
NPDSKLTLILVKNGGIVNGYVTLMGASDYVNTLFKNKNVSINVELYFDAT
GHILPDSSSLKTDLELKYKQTADFSARGFMPSTTAYPFVLPNAGTHNENY
IFGQCYYKASDGALFPLEVTVMLNKRLPDSRTSYVMTFLWSLNAGLAPET
TQATLITSPFTFSYIREDD
SEQ ID NO:21 is the 323 amino acid sequence for
the Ad35 fiber protein from GenBank Accession
No. AAA75331.
MTKRVRLSDSFNPVYPYEDESTSQHPFYNPGFISPNGFTQSPDGVLTLKC
LTPLTTTGGSLQLKVGGGLTVDDTDGTLQENIRATAPITKNNHSVELSIG
NGLETQNNKLCAKLGNGLKFNNGDICIKDSINTLWTGINPPPNCQIVENT
NTNDGKLTLVLVKNGGLVNGYVSLVGVSDTVNQMFTQKTANIQLRLYFDS
SGNLLTEESDLKIPLKNKSSTATSETVASSKAFMPSTTAYPFNTTTRDSE
NYIHGICYYMTSYDRSLFPLNISIMLNSRMISSNVAYAIQFEWNLNASES
PESNIATLTTSPFFFSYITEDDN

What is claimed is:

1. A method of transferring a heterologous nucleotide sequence into tumor cells, comprising: transducing said tumor cells with a modified adenovirus comprising at least one heterologous DNA sequence, wherein prior to modification said adenovirus is a Subgenus C adenovirus, and said modification comprises replacement of at least a portion of the fiber of said Subgenus C adenovirus with at least a portion of the fiber of an adenovirus of a second serotype, and wherein said tumor cells include a receptor which binds to said at least a portion of the fiber of said adenovirus of said second serotype, and whereby transfer of said at least one heterologous DNA sequence into said cells is effected through binding of said modified adenovirus fiber to said tumor cells.

2. The method of claim 1 wherein the fiber of said modified adenovirus includes a head region, a shaft region, and a tail region, and at least a portion of the head region of the fiber of said Subgenus C adenovirus is removed and replaced with at least a portion of the head region of the fiber of said adenovirus of said second serotype.

3. The method of claim 2 wherein said adenovirus of said second serotype is an adenovirus of a serotype within a subgenus selected from the group consisting of Subgenera A, B, D, E, and F.

4. The method of claim 3 wherein said adenovirus of said second serotype is an adenovirus of a serotype within Subgenus B.

5. The method of claim 4 wherein said adenovirus of said second serotype is Adenovirus 35.

6. The method of claim 5, wherein the shaft region of the fiber of said modified adenovirus is from Adenovirus 5, and the head region is from Adenovirus 35 and comprises amino acids 137 to 323 of SEQ ID NO:14 or SEQ ID NO:21.

7. The method of claim 5, wherein the Adenovirus 5 shaft region of the fiber of said modified adenovirus comprises amino acids 47 to 399 of SEQ ID NO:16

8. The method of claim 6, wherein the nucleotide sequence encoding the open reading frame (ORF) for the Adenovirus 5 shaft region and the Adenovirus 35 fiber region of said modified adenovirus comprises the sequence presented as SEQ ID NO:17.

9. The method of claim 6, wherein the amino acid sequence of the open reading frame (ORF) for the Adenovirus 5 shaft region and the Adenovirus 35 fiber head region of said modified adenovirus comprises the sequence presented as SEQ ID NO:18.

10. The method of claim 5, wherein the Adenovirus 5 shaft region of said modified adenovirus fiber comprises the KKTK sequence presented as SEQ ID NO:9.

11. The method of claim 10, wherein the KKTK sequence of said Adenovirus 5 fiber shaft sequence is deleted or mutated.

12. The method of claim 5, wherein the Adenovirus 5 shaft region of said modified adenovirus fiber comprises the KLGTGLSFD sequence presented as SEQ ID NO:10.

13. The method of claim 5, wherein the Adenovirus 5 shaft region of said modified adenovirus fiber comprises the GNLTSQNVTTVSPPLKKTK sequence presented as SEQ ID NO:11.

14. The method of claim 5, wherein said modified adenovirus comprises the E2F promoter having the sequence presented as SEQ ID NO:1.

15. The method of claim 5, wherein said modified adenovirus comprises the TERT promoter having the sequence presented as SEQ ID NO:2 or SEQ ID NO:3.

16. The method of claim 5, wherein said cells are selected from the group consisting of epidermoid cells, tongue cells, pharyngeal cells, nasal septum cells, skin cells and tumor cells including primary tumor cells and tumor cell lines.

17. The method of claim 5, wherein said cells are transduced with said modified adenovirus in vivo.

18. The method of claim 16, wherein said tumor cells are selected from the group consisting of epidermoid carcinoma cells, squamous cell carcinoma (SQCC) cells, tongue SQCC cells, pharyngeal carcinoma cells, nasal septum SQCC cells and skin malignant melanoma cells.

19. The method of claim 16, wherein said tumor cells are head and neck cancer cells.

20. The method of claim 16, wherein said tumor cells are melanoma cells.

21. The method of claim 5, wherein said heterologous DNA sequence encodes GM-CSF.

22. An adenovirus composition comprising an adenovirus with a modified fiber portion, comprising a fiber shaft region of an adenovirus of Subgenus C and a fiber head region from an Adenovirus 35 fiber, wherein said adenovirus exhibits a higher transduction efficiency for a cell which expresses relatively high levels of CD46 as compared to the transduction efficiency exhibited by an adenovirus of Subgenus C having an unmodified fiber.

23. The adenovirus composition of claim 22, wherein the shaft region of said modified fiber is from Adenovirus 5, and the head region is from Adenovirus 35 and comprises amino acids 137 to 323 of SEQ ID NO:14 or SEQ ID NO:21.

24. The adenovirus composition of claim 22, wherein the Adenovirus 5 shaft region of said modified fiber comprises amino acids 47 to 399 of SEQ ID NO:16

25. The adenovirus composition of claim 22, wherein the nucleotide sequence encoding the open reading frame (ORF) for the Adenovirus 5 shaft region and the Adenovirus 35 head region of said modified fiber is presented as SEQ ID NO:17.

26. The adenovirus composition of claim 22, wherein the amino acid sequence of the Adenovirus 5 shaft region and the Adenovirus serotype 35 fiber head region of said modified fiber is presented as SEQ ID NO:18.

27. The adenovirus composition of claim 22, wherein the Adenovirus 5 shaft region of said modified fiber comprises the KKTK sequence presented as SEQ ID NO:9.

28. The adenovirus composition of claim 27, wherein the KKTK sequence of said Adenovirus 5 shaft is deleted or mutated.

29. The adenovirus composition of claim 22, wherein the Adenovirus 5 shaft region of said modified fiber comprises the KLGTGLSFD sequence presented as SEQ ID NO:10.

30. The adenovirus composition of claim 22, wherein the Adenovirus 5 shaft region of said modified fiber comprises the GNLTSQNVTTVSPPLKKTK sequence presented as SEQ ID NO:11.

31. The adenovirus composition of claim 22, wherein said adenovirus comprises the E2F promoter having the sequence presented as SEQ ID NO:1.

32. The adenovirus composition of claim 22, wherein said adenovirus comprises the TERT promoter having the sequence presented as SEQ ID NO:2 or SEQ ID NO:3.

33. The adenovirus composition of claim 22, further comprising a heterologous DNA sequence encoding GM-CSF
